(19) United States
(12) Patent Application Publication
(10) Pub. No.: US 2023/0186115 A1
(43) Pub. Date: Jun. 15, 2023

(54) MACHINE LEARNING MODELS FOR DATA DEVELOPMENT AND PROVIDING USER INTERACTION POLICIES

(71) Applicant: Dexcom, Inc., San Diego, CA (US)

(72) Inventors: Afshan A. KLEINHANZL, San Diego, CA (US); Alexander Michael DIENER, San Diego, CA (US); Adam G. NOAR, JR., San Diego, CA (US); Stacey Lynne FISCHER, San Diego, CA (US); Chad M. PATTERSON, San Diego, CA (US); Carly Rose OLSON, San Diego, CA (US); Michiko Araki KELLEY, San Diego, CA (US); Amit Premal JOSHIPURA, San Diego, CA (US); Spencer Troy FRANK, San Diego, CA (US); Qi AN, San Diego, CA (US); Abdulrahman JBAILY, San Diego, CA (US); Sophia PARK, San Diego, CA (US); Justin Yi-Kai LEE, San Diego, CA (US); Joost Herman VAN DER LINDEN, San Diego, CA (US); Mark DERDZINSKI, Pittsburgh, PA (US)

(21) Appl. No.: 18/066,254

(22) Filed: Dec. 14, 2022

Related U.S. Application Data
(60) Provisional application No. 63/289,376, filed on Dec. 14, 2021.

(51) Int. Cl. G06N 5/022

(52) U.S. Cl. CPC G06N 5/022

(57) ABSTRACT

Systems, devices, and methods for data collection and development as well as providing user interaction policies are provided. In one embodiment, a method includes collecting contextual data for a first subset of a plurality of users. The method further includes generating a first set of contextual profiles for the first subset of the plurality of users based on the collected contextual data. Additionally, the method includes training one or more imputation models to develop the contextual data for the second subset of the plurality of users. The method also includes generating the contextual data for the second subset of the plurality of users using the one or more imputation models. Further, the method includes generating a second set of contextual profiles for the second subset of the plurality of users based on the generated contextual data for the second subset of the plurality of users.

### CROSS-REFERENCE TO RELATED APPLICATION

This application claims priority to U.S. Provisional Patent Application No. 63/289,376, filed on Dec. 14, 2021, the disclosure of which is incorporated herein by reference.

### FIELD OF DISCLOSURE

This application generally relates to medical devices (e.g., analyte sensors), and more specifically to systems, devices, and methods for data collection and development as well as providing user interaction policies.

### BACKGROUND

Diabetes is a metabolic condition relating to the production or use of insulin by the body. Insulin is a hormone that allows the body to use glucose for energy, or store glucose as fat.

When a person eats a meal that contains carbohydrates, the food is processed by the digestive system, which produces glucose in the person's blood. Blood glucose can be used for energy or stored as fat. The body normally maintains blood glucose levels in a range that provides sufficient energy to support bodily functions and avoids problems that can arise when glucose levels are too high, or too low. Regulation of blood glucose levels depends on the production and use of insulin, which regulates the movement of blood glucose into cells.

When the body does not produce enough insulin, or when the body is unable to effectively use insulin that is present, blood sugar levels can elevate beyond normal ranges. The state of having a higher than normal blood sugar level is called “hyperglycemia.” Chronic hyperglycemia can lead to a number of health problems, such as cardiovascular disease, cataract and other eye problems, nerve damage (neuropathy), and kidney damage. Hyperglycemia can also lead to acute problems, such as diabetic ketoacidosis—a state in which the body becomes excessively acidic due to the presence of blood glucose and ketones, which are produced when the body cannot use glucose. The state of having lower than normal blood glucose levels is called “hypoglycemia.” Severe hypoglycemia can lead to acute crises that can result in seizures or death.

A diabetes patient can receive insulin to manage blood glucose levels. Insulin can be received, for example, through a manual injection with a needle. Wearable insulin pumps may also be utilized to receive insulin. Diet and exercise also affect blood glucose levels.

Diabetes conditions may be referred to as “Type 1” and “Type 2.” A Type 1 diabetes patient is typically able to use insulin when it is present, but the body is unable to produce sufficient amounts of insulin, because of a problem with the insulin-producing beta cells of the pancreas. A Type 2 diabetes patient may produce some insulin, but the patient has become “insulin resistant” due to a reduced sensitivity to insulin. The result is that even though insulin is present in the body, the insulin is not sufficiently used by the patient's body to effectively regulate blood sugar levels

For diabetes patients, monitoring blood glucose levels and regulating those levels to be within an acceptable range is important not only to mitigate long-term issues such as heart disease and vision loss, but also to avoid the effects of hyperglycemia and hypoglycemia. Maintaining blood glucose levels within an acceptable range can be challenging, as this level is almost constantly changing over time and in response to everyday events, such as eating or exercising. Advances in medical technologies have enabled development of various systems for monitoring blood glucose, including continuous glucose monitoring (CGM) systems, which measure and record glucose concentrations in substantially real-time. CGM systems are important tools for users of these systems to ensure that measured glucose values are within the acceptable range.

This background is provided to introduce a brief context for the summary and detailed description that follow. This background is not intended to be an aid in determining the scope of the claimed subject matter nor be viewed as limiting the claimed subject matter to implementations that solve any or all of the disadvantages or problems presented above.

### BRIEF SUMMARY

The various embodiments of the present systems, devices, and methods comprise several features, no single one of which is solely responsible for their desirable attributes. Without limiting the scope of the present embodiments, their more prominent features will now be discussed below. After considering this discussion, and particularly after reading the section entitled “Detailed Description,” one will understand how the features of the present embodiments provide the advantages described here.

In a first aspect, a method comprises: dividing a plurality of users into an exploration subset of users and an exploitation subset of users; randomly assigning at least one user interaction policy to each of the exploration subset of users; and determining at least one user interaction policy for each of the exploitation subset of users using one or more contextual models trained using contextual data corresponding to the exploitation subset of users, wherein the contextual data corresponding to the exploitation subset of users comprises at least some of a first set of contextual profiles and a second set of contextual profiles.

In a second aspect, a method comprises collecting contextual data for a first subset of a plurality of users; generating a first set of contextual profiles for the first subset of the plurality of users based on the collected contextual data; determining that contextual data for a second subset of the plurality of users is incomplete or not available; training one or more imputation models based on the contextual data for the first subset of the plurality of users to develop the contextual data for the second subset of the plurality of users; generating the contextual data for the second subset of the plurality of users using the one or more imputation models; and generating the second set of contextual profiles for the second subset of the plurality of users based on the generated contextual data for the second subset of the plurality of users.

Also described herein are embodiments of a non-transitory computer readable medium comprising instructions to be executed in a computer system, wherein the instructions when executed in the computer system perform the methods described above.

Also described herein are embodiments of a computer system, wherein software for the computer system is programmed to execute the methods described above.

Also described herein are embodiments of a computer system comprising means for executing the methods described above.

### BRIEF DESCRIPTION OF THE DRAWINGS

FIG. 1 is an illustration of an environment in an example implementation that is operable to employ techniques described herein, in accordance with certain embodiments of the disclosure.

FIG. 2 depicts an example of the analyte monitoring device of FIG. 1 in greater detail, in accordance with certain embodiments of the disclosure.

FIG. 3 is a flow diagram illustrating a process for determining user interaction policies based on psychographics, in accordance with certain embodiments of the disclosure.

FIG. 4 is an exemplary operational process for performing the psychographic data collection and development phase and the exploration-exploitation phase of the process of FIG. 3, in accordance with certain embodiments of the disclosure.

FIG. 5 depicts an example of an achievement user interface (“UI”) displayed, in accordance with certain embodiments of the disclosure.

FIG. 6 depicts another example of achievement Uls displayed, in accordance with certain embodiments of the disclosure.

FIG. 7 depicts another example of an achievement UI displayed, in accordance with certain embodiments of the disclosure.

FIG. 8 depicts an example of badges displayed via achievement Uls of a user and via achievement Uls of additional users, in accordance with certain embodiments of the disclosure.

FIG. 9 depicts an example of family united Uls displayed, in accordance with certain embodiments of the disclosure.

FIG. 10 depicts another example of family united UIs displayed, in accordance with certain embodiments of the disclosure.

FIG. 11 depicts another example of a family united UI displayed, in accordance with certain embodiments of the disclosure.

FIG. 12 depicts another example of a family united UI displayed, in accordance with certain embodiments of the disclosure.

FIG. 13 depicts an example of a journey together UI displayed, in accordance with certain embodiments of the disclosure.

FIG. 14 depicts another example of a journey together UI displayed, in accordance with certain embodiments of the disclosure.

FIG. 15 depicts another example of a journey together UI displayed, in accordance with certain embodiments of the disclosure.

FIG. 16 depicts an example of a glucose inspiration UI displayed, in accordance with certain embodiments of the disclosure.

FIG. 17 depicts another example of a glucose inspiration UI displayed, in accordance with certain embodiments of the disclosure.

FIG. 18 depicts another example of a glucose inspiration UI displayed, in accordance with certain embodiments of the disclosure.

FIG. 19 depicts an example of a follower highlights UI displayed, in accordance with certain embodiments of the disclosure.

FIG. 20 depicts another example of a follower highlights UI displayed, in accordance with certain embodiments of the disclosure.

FIG. 21 depicts another example of a follower highlights UI displayed, in accordance with certain embodiments of the disclosure.

FIG. 22 depicts an example of an expert portal UI displayed, in accordance with certain embodiments of the disclosure.

FIG. 23 depicts another example of an expert portal UI displayed, in accordance with certain embodiments of the disclosure.

FIG. 24 depicts another example of an expert portal UI displayed, in accordance with certain embodiments of the disclosure.

FIG. 25 depicts an example of personalization Uls displayed, in accordance with certain embodiments of the disclosure.

FIG. 26 depicts an example of an appointment prep UI displayed, in accordance with certain embodiments of the disclosure.

FIG. 27 depicts another example of an appointment prep UI displayed, in accordance with certain embodiments of the disclosure.

FIG. 28 depicts another example of an appointment prep UI displayed, in accordance with certain embodiments of the disclosure.

FIG. 29 depicts another example of an appointment prep UI displayed, in accordance with certain embodiments of the disclosure.

FIG. 30 depicts another example of an appointment prep UI displayed, in accordance with certain embodiments of the disclosure.

FIG. 31 depicts another example of an appointment prep UI displayed, in accordance with certain embodiments of the disclosure.

FIG. 32 depicts an example of a video stories UI displayed, in accordance with certain embodiments of the disclosure.

FIG. 33 depicts another example of a video stories UI displayed, in accordance with certain embodiments of the disclosure.

FIG. 34 depicts another example of a video stories UI displayed, in accordance with certain embodiments of the disclosure.

FIG. 35 depicts another example of a video stories UI displayed, in accordance with certain embodiments of the disclosure.

FIG. 36 depicts an example of a health hub UI displayed, in accordance with certain embodiments of the disclosure.

FIG. 37 depicts another example of a health hub UI displayed, in accordance with certain embodiments of the disclosure.

FIG. 38 depicts another example of a health hub UI displayed, in accordance with certain embodiments of the disclosure.

FIG. 39 illustrates an example system including various components of an example device that can be implemented as any type of computing device as described and/or utilized with reference to FIGS. 1-38 to implement embodiments of the techniques described herein, in accordance with certain embodiments of the disclosure.

### DETAILED DESCRIPTION

Portable and/or wearable health monitoring devices (also referred to herein as “health monitoring devices”) and mobile health applications (also referred to herein as “applications”), have rapidly become renowned for their capabilities to support user-centered care. For example, management of diabetes can present complex challenges for patients, clinicians, and caregivers, as a confluence of many factors can impact a patient's glucose level and glucose trends. To assist patients with better managing this condition, health monitoring devices (e.g., sensors and other types of monitoring and diagnostic devices) as well as a variety of mobile health applications such as, but not limited to, health and fitness monitoring applications, which can assist with diabetes management for type 1 and type 2 diabetes patients have been developed. The wide dissemination of health monitoring devices and the increase in the development and distribution of mobile health applications has improved health management, and more specifically chronic disease management, in the healthcare domain. In particular, the use of mobile health applications in conjunction with these health monitoring devices, represents a more scalable and potentially more cost-effective alternative to traditional interventions, offering a means of improving health and chronic disease management by expanding the reach of healthcare services and improving users' access to health-related information and interventions.

Mobile health applications enable users to be much more involved in the users' own medical care by granting them access to and control over their health information. In particular, mobile health applications enable users to access, monitor, record, and update their health information regardless of physical constraints, such as time and location. In particular, a variety of intervention applications have been developed to deliver guidance that may assist patients, caregivers, healthcare providers, or other users in improving lifestyle or clinical/patient outcomes by meeting a variety of challenges, such as analyte control, exercise, and/or other health factors. For example, diabetes intervention applications may assist patients, caregivers, healthcare providers, or other users in overnight glucose control (e.g., reduce incidence of hypoglycemic events or hyperglycemic excursions), glucose control during and after meals (e.g., use historical information and trends to increase glycemic control), hyperglycemia corrections (e.g., increase time in target zone while avoiding hypoglycemic events from over-correction), and/or hypoglycemia treatments (e.g., address hypoglycemia while avoiding “rebound” hyperglycemia), to name a few.

Unfortunately, many mobile health applications designed to support the management of chronic diseases or health conditions have been plagued with low user engagement and high user attrition rates. A reason for low user engagement and/or high user attrition rates may include failure of mobile health applications to provide individualized or personalized user interaction policies (as defined further below) to the users. When a mobile health application fails to provide individualized and/or personalized user interaction policies, users of the application may find the interaction to be ineffective in enabling them to take a holistic approach to managing their health (e.g., diseases, conditions, fitness, etc.). Further, user interaction policies that are not tailored to an individual (i.e., not “individualized”) may result in sub-optimal health outcomes. In addition, user interaction policies that are not tailored to a cohort that the individual is a member (i.e., not “personalized”) may also result in sub-optimal health outcomes. Furthermore, user interaction polices that are not individualized and/or personalized, may result in user interaction policies that are not relevant to the user. Thus, user engagement associated with such mobile health applications may decrease and, thereby, user attrition rates may increase.

Accordingly, certain embodiments described herein are directed to a health monitoring platform that uses contextual multi-armed bandit (MAB) models to provide individualized or personalized user interaction policies to users, for example, based on their corresponding contextual data. User interaction policies refer to the various ways or means through which the software application interacts with users. For example, user interaction policies may include recommendations (e.g., increase your steps, get more sleep, you might be interested in intermittent fasting, etc.), insights, content, information, etc., provided to a user. User interaction policies may also include delivery methods (e.g., video, audio, text, phone call, emails, push notification, in app messages, etc.) for delivering such recommendations, insights, content, information, etc. to the user. User interaction policies may further include UI configurations associated with, for example, different layouts of the software application used by the user. An objective of the software application is to individualize or personalize the user interaction policies for each user by selecting and providing, to each user, user interaction policies that maximize user engagement by that specific user and/or the likelihood that the specific user achieves their goals.

In order to achieve this level of individualization and/or personalization of the user interaction policies, the health monitoring platform may utilize contextual MAB models that take, as input, contextual data corresponding to a user, and output individualized and/or personalized user interaction policies to each user. A user's contextual data may include the user's demographic information (e.g., age, gender, ethnicity, etc.), physiological information (e.g., analyte information generated by one or more analyte sensors), non-analyte health information (e.g., heart rate, temperature, or other data generated by non-analyte sensors), disease information, and any other health related information), and any other relevant user-specific information.

A user's contextual data also includes psychographic data. As used herein, psychographic data may include various data related to goals, interests, values, attitudes, personality traits, behavior related data, etc. For example, user goals may include sleep goals, exercise goals, eating goals, glucose management goals, etc.). User interests may include interested in certain types of exercises, certain types of activities, etc. Having access to a user's psychographic data plays an important role in providing individualized or personalized user interaction policies to users that maximize user engagement. If a user's goal is unknown, user interaction policies (e.g., recommendations) provided to the user may not be beneficial in helping the user achieve the goal. For example, if a diabetic user's goal of getting uninterrupted sleep is unknown by the health monitoring platform, providing recommendations that help the user achieve that goal may be unlikely. Similarly, if a user's interest in running (as opposed to other types of exercise) is unknown by the health monitoring platform, recommending that the user engage in weightlifting to achieve a weight loss goal may be unhelpful to the user. Thus, obtaining user's psychographic data and utilizing it as part of the contextual data that is used as input into contextual MAB models for providing user interaction policies is advantageous.

However, there is a technical challenge associated with obtaining psychographic data, as it may not always be fully available for each user. For example, unlike demographic and/or physiological information which may be available for a new user who just started using the health monitoring platform, psychographic data may not be available. Further, even when users are asked to provide psychographic data, they may not necessarily be responsive to such requests. At the same time, overburdening users to provide inputs by continuously, or frequently, seeking such information may not even be desirable, as it results in a negative experience and thus higher user attrition rates. Further, continuously asking a user to provide input about their psychographic data and updating such information for processing may be technically disadvantageous, as such a process would use the limited processing, memory, battery, and network resources that are available to the computing device used by the user.

As such, certain embodiments herein are directed to performing a psychographic data collection and development phase that collects psychographic data for a plurality of users as well as trains imputations models for inferring user psychographic data that is not available for other users, as further described below. Obtaining psychographic data about users allows for creating a more complete training dataset that can then be used in training the contextual MAB models, discussed above, for determining user interaction policies during a later exploration-exploitation phase.

By utilizing imputation models, the psychographics data collection and development phase is able to generate psychographic data that is not available and provide a more useful and complete training data for use by the contextual MAB models. In particular, the imputation models may be used to infer psychographic data for new users and/or for users where information is not available or limited. For example, for a new user, while demographic information may be available, information about the user's goals and/or interests may not be available. For each of a plurality of psychographic features (e.g., goals, behaviors (e.g., diabetic distress, fear of hyperglycemia, psychological well-being), interests, concerns, psychological well-being, etc.) that may be used by the exploration-exploitation to provide user interaction policies, there may be a different imputation model for inferring the corresponding value for the psychographic feature, as further discussed below.

The systems, devices, and methods of the embodiments described herein can be used in conjunction with any type of analyte sensor for any measurable analyte. Further, the system, devices, and methods of the embodiment described herein may be used in conjunction with any health-related application that is provided to the user to improve the user's health. For example, a health-related application may help the user with treating a certain disease or just help with improving the overall health of a user who is not necessarily diagnosed with a disease.

### Example Environment with a Data Analytics Platform for Determining User Interaction Policies based on User Psychographics

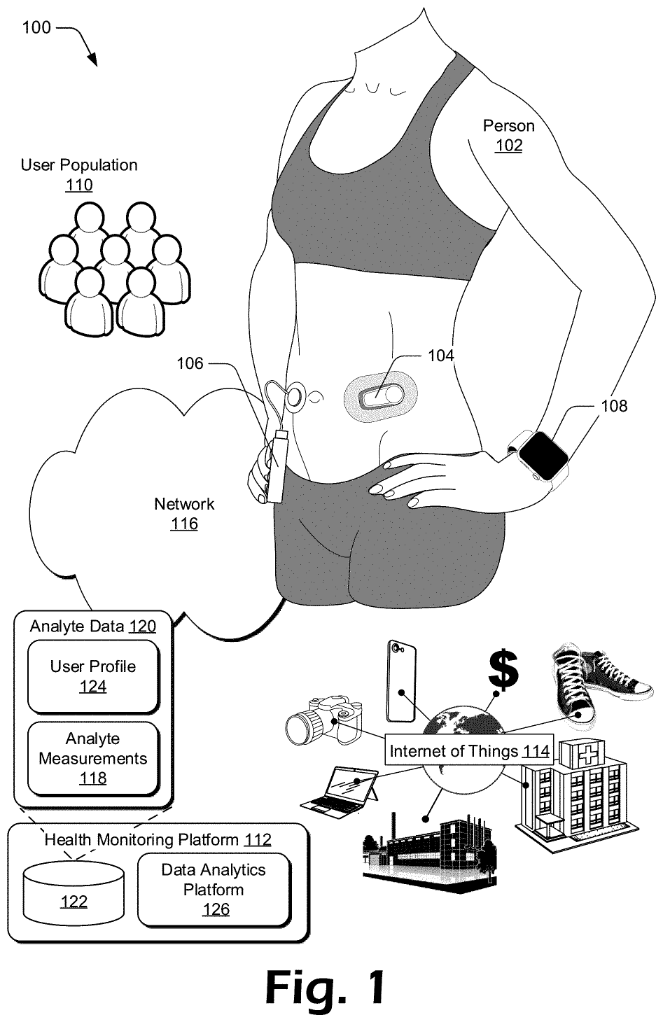

FIG. 1 is an illustration of an environment 100 in an example implementation that is operable to employ techniques as described herein. The illustrated environment 100 includes person 102, who is depicted wearing an analyte monitoring device 104, medicament delivery system 106, and computing device 108. The illustrated environment 100 also includes other users in a user population 110 of the analyte monitoring device, health monitoring platform 112, and Internet of Things 114 (IoT 114). The analyte monitoring device 104, medicament delivery system 106, computing device 108, user population 110, health monitoring platform 112, and IoT 114 are communicatively coupled, one to another, via a network 116.

Alternately or additionally, one or more of the analyte monitoring device 104, the medicament delivery system 106, and the computing device 108 may be communicatively coupled in other ways, such as using one or more short range communication protocols or techniques. For example, the analyte monitoring device 104, the medicament delivery system 106, and the computing device 108 may communicate with one another using one or more of Bluetooth, near-field communication (NFC), 5G, and so forth. The analyte monitoring device 104, the medicament delivery system 106, and the computing device 108 may leverage these types of communication to form a closed-loop system between one another. In this way, the medicament delivery system 106 may deliver a medicament (e.g., insulin) based on predictions of an analyte level (e.g., glucose level) computed in real-time (e.g., by the computing device 108) as analyte measurements are obtained by the analyte monitoring device 104.

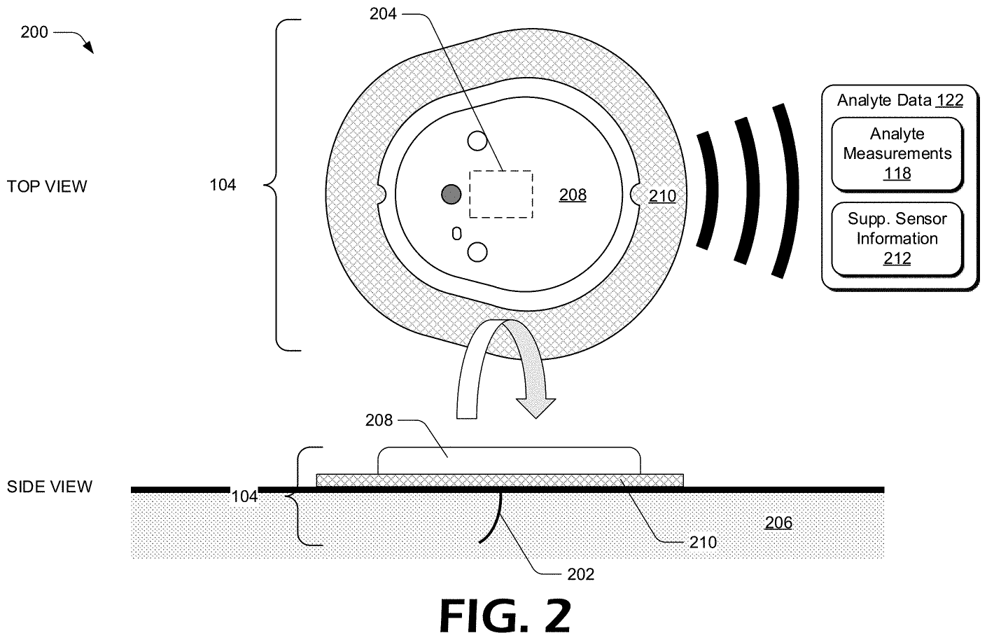

In one or more implementations, the analyte monitoring device 104 is a continuous glucose monitoring (“CGM”) device. As used herein, the term “continuous” when used in connection with analyte monitoring may refer to an ability of a device to produce measurements substantially continuously, such that the device may be configured to produce the analyte measurements at regular or irregular intervals of time (e.g., approximately every hour, approximately every 30 minutes, approximately every 5 minutes, and so forth), responsive to establishing a communicative coupling with a different device (e.g., when the computing device 108 establishes a wireless connection with the analyte monitoring device 104 to retrieve one or more of the measurements), and so forth. This functionality along with further aspects of the configuration of the analyte monitoring device 104 are discussed in more detail in relation to FIG. 2.

In one or more implementations, the analyte monitoring device 104 transmits the analyte measurements 118 to one or more of the computing device 108, the medicament delivery system 106, or some other device, such as via Bluetooth. The analyte monitoring device 104 may communicate these measurements in real-time, e.g., as they are produced using an analyte sensor. Alternately or in addition, the analyte monitoring device 104 may communicate the analyte measurements 118 at set time intervals, e.g., every 30 seconds, every minute, every 5 minutes, every hour, every 6 hours, every day, and so forth. Further still, the analyte monitoring device 104 may communicate these measurements responsive to a request for the measurements, e.g., communicated to the analyte monitoring device 104 when the computing device 108 causes display of a user interface having information about the person 102's glucose level, updates such a display, predicts the person 102's upcoming glucose level for the purpose of delivering insulin, and so forth. Accordingly, the computing device 108 may maintain the analyte measurements 118 of the person 102 at least temporarily, e.g., in computer readable storage media of the computing device 108.

Note that, while in certain examples the analyte monitoring device 104 is assumed to be a glucose monitoring device, analyte monitoring device 104 may operate to monitor one or more additional or alternative analytes. The term “analyte” as used herein is a broad term, and is to be given its ordinary and customary meaning to a person of ordinary skill in the art (and is not to be limited to a special or customized meaning), and refers without limitation to a substance or chemical constituent in the body or a biological sample (e.g., bodily fluids, including, blood, serum, plasma, interstitial fluid, cerebral spinal fluid, lymph fluid, ocular fluid, saliva, oral fluid, urine, excretions, or exudates). Analytes can include naturally occurring substances, artificial substances, metabolites, and/or reaction products. In some embodiments, the analyte for measurement by the sensing regions, devices, and methods is albumin, alkaline phosphatase, alanine transaminase, aspartate aminotransferase, bilirubin, blood urea nitrogen, calcium, CO2, chloride, creatinine, glucose, gamma-glutamyl transpeptidase, hematocrit, lactate, lactate dehydrogenase, magnesium, oxygen, pH, phosphorus, potassium, sodium, total protein, uric acid, metabolic markers, and drugs.

Other analytes are contemplated as well, including but not limited to acetaminophen, dopamine, ephedrine, terbutaline, ascorbate, uric acid, oxygen, d-amino acid oxidase, plasma amine oxidase, xanthine oxidase, NADPH oxidase, alcohol oxidase, alcohol dehydrogenase, pyruvate dehydrogenase, diols, Ros, NO, bilirubin, cholesterol, triglycerides, gentisic acid, ibuprophen, L-Dopa, methyl dopa, salicylates, tetracycline, tolazamide, tolbutamide, acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); biotinidase; biopterin; c-reactive protein; carnitine; carnosinase; CD4; ceruloplasmin; chenodeoxycholic acid; chloroquine; cholesterol; cholinesterase; conjugated 1-β hydroxy-cholic acid; cortisol; creatine kinase; creatine kinase MM isoenzyme; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, glucose-6-phosphate dehydrogenase, hemoglobin A, hemoglobin S, hemoglobin C, hemoglobin D, hemoglobin E, hemoglobin F, D-Punjab, beta-thalassemia, hepatitis B virus, HCMV, HIV-1, HTLV-1, Leber hereditary optic neuropathy, MCAD, RNA, PKU, Plasmodium vivax, sexual differentiation, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free (3-human chorionic gonadotropin; free erythrocyte porphyrin; free thyroxine (FT4); free tri-iodothyronine (FT3); fumarylacetocetase; galactose/gal-1-phosphate; galactose-1-phosphate uridyltransferase; gentamicin; glucose-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; lead; lipoproteins ((a), B/A-1, β); lysozyme; mefloquine; netilmicin; phenobarbitone; phenyloin; phytanic/pristanic acid; progesterone; prolactin; prolidase; purine nucleoside phosphorylase; quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin. Salts, sugar, protein, fat, vitamins, and hormones naturally occurring in blood or interstitial fluids can also constitute analytes in certain embodiments.

The analyte can be naturally present in the biological fluid, for example, a metabolic product, a hormone, an antigen, an antibody, and the like. Alternatively, the analyte can be introduced into the body, for example, a contrast agent for imaging, a radioisotope, a chemical agent, a fluorocarbon-based synthetic blood, or a drug or pharmaceutical composition, including but not limited to insulin; ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbituates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The metabolic products of drugs and pharmaceutical compositions are also contemplated analytes. Analytes such as neurochemicals and other chemicals generated within the body can also be analyzed, such as, for example, ascorbic acid, uric acid, dopamine, noradrenaline, 3-methoxytyramine (3MT), 3,4-dihydroxyphenylacetic acid (DOPAC), homovanillic acid (HVA), 5-hydroxytryptamine (5HT), histamine, Advanced Glycation End Products (AGEs) and 5-hydroxyindoleacetic acid (FHIAA).

Although illustrated as a wearable device (e.g., a smart watch), the computing device 108 may be configured in a variety of ways without departing from the spirit or scope of the described techniques. By way of example and not limitation, the computing device 108 may be configured as a different type of mobile device (e.g., a mobile phone, tablet device, smart ring, or other wearable device, the computing device 108 may be configured as a dedicated device associated with the health monitoring platform 112, e.g., with functionality to obtain the analyte measurements 118 from the analyte monitoring device 104, perform various computations in relation to the analyte measurements 118, display information related to the analyte measurements 118 and the health monitoring platform 112, communicate the analyte measurements 118 to the health monitoring platform 112, and so forth. In contrast to implementations where the computing device 108 is configured as a mobile phone, however, the computing device 108 may not include some functionality available with mobile phone or wearable configurations when configured as a dedicated analyte monitoring device, such as the ability to make phone calls, camera functionality, the ability to utilize social networking applications, and so on.

Additionally, the computing device 108 may be representative of more than one device in accordance with the described techniques. In one or more scenarios, for instance, the computing device 108 may correspond to both a wearable device (e.g., a smart watch) and a mobile phone. In such scenarios, both of these devices may be capable of performing at least some of the same operations, such as to receive the analyte measurements 118 from the analyte monitoring device 104, communicate them via the network 116 to the health monitoring platform 112, display information related to the analyte measurements 118, and so forth.

Alternately or in addition, different devices may have different capabilities that other devices do not have or that are limited through computing instructions to specified devices. In the scenario where the computing device 108 corresponds to a separate smart watch and a mobile phone, for instance, the smart watch may be configured with various sensors and functionality to measure a variety of physiological markers (e.g., heartrate, breathing, rate of blood flow, and so on) and activities (e.g., steps, movement, etc.) of the person 102. In this scenario, the mobile phone may not be configured with these sensors and functionality or may include a limited amount of that functionality—although in other scenarios a mobile phone may be able to provide the same functionality. Continuing with this particular scenario, the mobile phone may have capabilities that the smart watch does not have, such as an amount of computing resources (e.g., battery and processing speed) that enables the mobile phone to more efficiently carry out computations in relation to the analyte measurements 118. Even in scenarios where a smart watch is capable of carrying out such computations, computing instructions may limit performance of those computations to the mobile phone so as not to burden both devices and to utilize available resources efficiently. To this extent, the computing device 108 may be configured in different way and represent different numbers of devices than discussed herein without departing from the spirit and scope of the described techniques.

As mentioned above, the computing device 108 communicates the analyte measurements 118 to the health monitoring platform 112. In the illustrated environment 100, the analyte measurements 118 are shown stored in storage device 122 of the health monitoring platform 112 as part of analyte data 120. The storage device 122 may represent one or more databases and also other type of storage capable of storing the analyte data 120. The analyte data 120 also includes user profile 124. In accordance with the described techniques, the person 102 corresponds to a user of at least the health monitoring platform 112 and may also be a user of one or more other, third-party service providers. To this end, the person 102 may be associated with a username and be required, at some time, to provide authentication information (e.g., password, biometric data, and so forth) to access the health monitoring platform 112 using the username. This information may be captured in the user profile 124. The user profile 124 may also include a variety of other information about the user, such as demographic information describing the person 102, information about a health care provider, payment information, prescription information, determined health indicators, user preferences, account information for other service provider systems (e.g., a service provider associated with a wearable, social networking systems, and so on), and so forth. The user profile 124 may include different information about a user within the spirit and scope of the described techniques.

Further, the analyte data 120 not only represents data of a user that corresponds to the person 102, but also represents data of the other users in the user population 110. Given this, the analyte measurements 118 in the storage device 122 include the analyte measurements from an analyte sensor of the analyte monitoring device 104 worn by the person 102 and also include analyte measurements from analyte sensors of analyte systems worn by persons corresponding to the other users in the user population 110. It follows also that the analyte measurements 118 of these other users are communicated by their respective devices via the network 116 to the health monitoring platform 112 and that these other users have respective user profiles 124 with the health monitoring platform 112.

The data analytics platform 126 represents functionality to process the analyte data 120 to generate a variety of predictions, such as by using various machine learning models. Based on these predictions, the health monitoring platform 112 may provide a recommended action. For instance, the health monitoring platform 112 may provide the decision support output directly to the user, to a medical professional associated with the user, and so forth. Although depicted as separate from the computing device 108, portions or an entirety of the data analytics platform 126 may alternately or additionally be implemented at the computing device 108. The data analytics platform 126 is also configured to generate these predictions using data in addition to the analyte measurements 118, such as additional data obtained via the IoT 114.

It is to be appreciated that the IoT 114 represents various sources capable of providing data that describes the person 102 and the person 102's activity as a user of one or more service providers and activity with the real world. By way of example, the IoT 114 may include various devices of the user, e.g., cameras, mobile phones, laptops, and so forth. To this end, the IoT 114 may provide information about interaction of the user with various devices, e.g., interaction with web-based applications, photos taken, communications with other users, and so forth. The IoT 114 may also include various real-world articles (e.g., shoes, clothing, sporting equipment, appliances, automobiles, etc.) configured with sensors to provide information describing behavior, such as steps taken, force of a foot striking the ground, length of stride, temperature of a user (and other physiological measurements), temperature of a user's environment, types of food stored in a refrigerator, types of food removed from a refrigerator, driving habits, and so forth.

The IoT 114 may also include third parties to the health monitoring platform 112, such as medical providers (e.g., a medical provider of the person 102) and manufacturers (e.g., a manufacturer of the analyte monitoring device 104, the medicament delivery system 106, or the computing device 108) capable of providing medical and manufacturing data, respectively, that can be leveraged by the data analytics platform 126. Certainly, the IoT 114 may include devices and sensors capable of providing a wealth of data in connection with recommendations based on analyte monitoring (e.g., continuous glucose monitoring) without departing from the spirit or scope of the described techniques. In the context of measuring an analyte, e.g., continuously, and obtaining data describing such measurements, consider the following discussion of FIG. 2.

FIG. 2 depicts an example 200 of an implementation of the analyte monitoring device 104 of FIG. 1 in greater detail, in accordance with certain embodiments of the disclosure. In particular, the illustrated example 200 includes a top view and a corresponding side view of the analyte monitoring device 104. It is to be appreciated that the analyte monitoring device 104 may vary in implementation from the following discussion in various ways without departing from the spirit or scope of the described techniques.

In this example 200, the analyte monitoring device 104 is illustrated to include an analyte sensor 202 (e.g., a glucose sensor) and a sensor module 204. Here, the analyte sensor 202 is depicted in the side view having been inserted subcutaneously into skin 206, e.g., of the person 102. The sensor module 204 is approximated in the top view as a dashed rectangle. The analyte monitoring device 104 also includes a transmitter 208 in the illustrated example 200. Use of the dashed rectangle for the sensor module 204 indicates that it may be housed or otherwise implemented within a housing of the transmitter 208. Antennae and/or other hardware used to enable the transmitter 208 to produce signals for communicating data, e.g., over a wireless connection to the computing device 108, may also be housed or otherwise implemented within the housing of the transmitter 208. In this example 200, the analyte monitoring device 104 further includes adhesive pad 210.

In operation, the analyte sensor 202 and the adhesive pad 210 may be assembled to form an application assembly, where the application assembly is configured to be applied to the skin 206 so that the analyte sensor 202 is subcutaneously inserted as depicted. In such scenarios, the transmitter 208 may be attached to the assembly after application to the skin 206 via an attachment mechanism (not shown). Alternatively, the transmitter 208 may be incorporated as part of the application assembly, such that the analyte sensor 202, the adhesive pad 210, and the transmitter 208 (with the sensor module 204) can all be applied substantially at once to the skin 206. In one or more implementations, this application assembly is applied to the skin 206 using a separate sensor applicator (not shown). Unlike the finger sticks required by conventional blood glucose meters, user-initiated application of the analyte monitoring device 104 with a sensor applicator is nearly painless and does not require the withdrawal of blood. Moreover, the automatic sensor applicator generally enables the person 102 to embed the analyte sensor 202 subcutaneously into the skin 206 without the assistance of a clinician or healthcare provider.

The analyte monitoring device 104 may also be removed by peeling the adhesive pad 210 from the skin 206. It is to be appreciated that the analyte monitoring device 104 and its various components as illustrated are simply one example form factor, and the analyte monitoring device 104 and its components may have different form factors without departing from the spirit or scope of the described techniques.

In operation, the analyte sensor 202 is communicably coupled to the sensor module 204 via at least one communication channel which can be a wireless connection or a wired connection. Communications from the analyte sensor 202 to the sensor module 204 or from the sensor module 204 to the analyte sensor 202 can be implemented actively or passively and these communications can be continuous (e.g., analog) or discrete (e.g., digital).

The analyte sensor 202 may be a device, a molecule, and/or a chemical which changes or causes a change in response to an event which is at least partially independent of the analyte sensor 202. The sensor module 204 is implemented to receive indications of changes to the analyte sensor 202 or caused by the analyte sensor 202. For example, the analyte sensor 202 can include glucose oxidase which reacts with glucose and oxygen to form hydrogen peroxide that is electrochemically detectable by the sensor module 204 which may include an electrode. In this example, the analyte sensor 202 may be configured as or include a glucose sensor configured to detect analytes in blood or interstitial fluid that are indicative of glucose level using one or more measurement techniques. In one or more implementations, the analyte sensor 202 may also be configured to detect analytes in the blood or the interstitial fluid that are indicative of other markers, such as lactate levels, ketones, or ionic potassium, which may improve accuracy in identifying or predicting glucose-based events. Additionally or alternatively, the analyte monitoring device 104 may include additional sensors and/or architectures to the analyte sensor 202 to detect those analytes indicative of the other markers.

In another example, the analyte sensor 202 (or an additional sensor of the analyte monitoring device 104 — not shown) can include a first and second electrical conductor and the sensor module 204 can electrically detect changes in electric potential across the first and second electrical conductor of the analyte sensor 202. In this example, the sensor module 204 and the analyte sensor 202 are configured as a thermocouple such that the changes in electric potential correspond to temperature changes. In some examples, the sensor module 204 and the analyte sensor 202 are configured to detect a single analyte, e.g., glucose. In other examples, the sensor module 204 and the analyte sensor 202 are configured to use diverse sensing modes to detect multiple analytes, e.g., ionic sodium, ionic potassium, carbon dioxide, and glucose. Alternatively or additionally, the analyte monitoring device 104 includes multiple sensors to detect not only one or more analytes (e.g., ionic sodium, ionic potassium, carbon dioxide, glucose, and insulin) but also one or more environmental conditions (e.g., temperature). Thus, the sensor module 204 and the analyte sensor 202 (as well as any additional sensors) may detect the presence of one or more analytes, the absence of one or more analytes, and/or changes in one or more environmental conditions. As noted above, the analyte monitoring device 104 may be configured to produce data describing a single analyte (e.g., glucose) or multiple analytes. Further, a combination of the analytes for which analyte monitoring devices are configured may vary across different lots of the monitoring devices manufactured (e.g., by the health monitoring platform 112), such that analyte monitoring devices having different architectures may be configured for use by different patient populations and/or for different health conditions.

In one or more implementations, the sensor module 204 may include a processor and memory (not shown). The sensor module 204, by leveraging the processor, may generate the analyte measurements 118 based on the communications with the analyte sensor 202 that are indicative of the above-discussed changes. Based on the above-noted communications from the analyte sensor 202, the sensor module 204 is further configured to generate communicable packages of data that include at least one analyte measurement 118. In this example 200, the analyte data 116 represents these packages of data. Additionally or alternatively, the sensor module 204 may configure the analyte data 116 to include additional data, including, by way of example, supplemental sensor information 212. The supplemental sensor information 212 may include a sensor identifier, a sensor status, temperatures that correspond to the analyte measurements 118, measurements of other analytes that correspond to the analyte measurements 118, and so forth. It is to be appreciated that supplemental sensor information 212 may include a variety of data that supplements at least one analyte measurement 118 without departing from the spirit or scope of the described techniques.

In implementations where the analyte monitoring device 104 is configured for wireless transmission, the transmitter 208 may transmit the analyte data 116 as a stream of data to a computing device. Alternatively or additionally, the sensor module 204 may buffer the analyte measurements 118 and/or the supplemental sensor information 212 (e.g., in memory of the sensor module 204 and/or other physical computer-readable storage media of the analyte monitoring device 104) and cause the transmitter 208 to transmit the buffered analyte data 120 later at various regular or irregular intervals, e.g., time intervals (approximately every second, approximately every thirty seconds, approximately every minute, approximately every five minutes, approximately every hour, and so on), storage intervals (when the buffered analyte measurements 118 and/or supplemental sensor information 212 reach a threshold amount of data or a number of measurements), when requested by another device, and so forth.

Having considered an example environment and example analyte monitoring system, consider now a discussion of some example details of the techniques in accordance with one or more implementations.

### Example Processes for Determining User Interaction Policies based on Psychographics

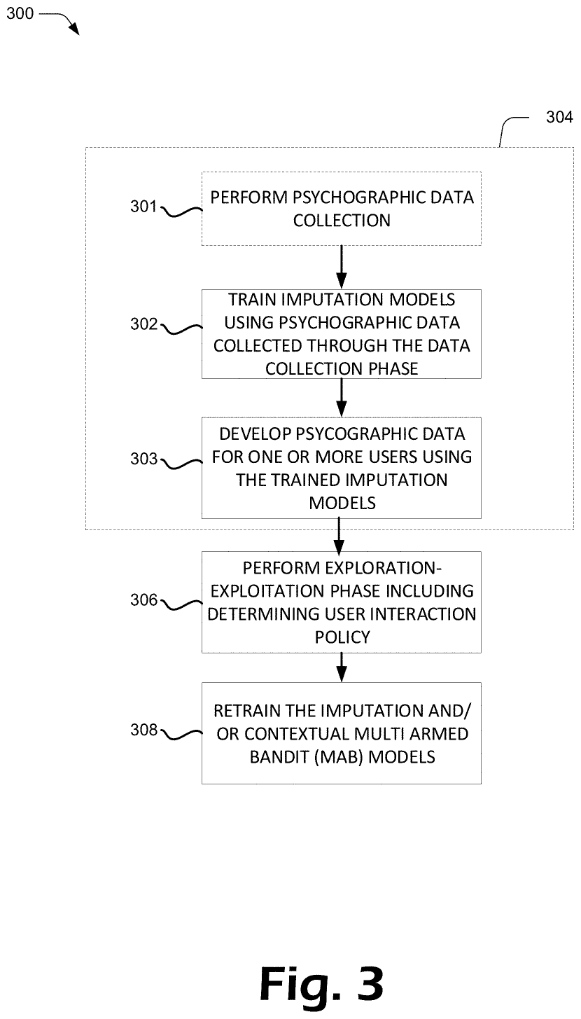

FIG. 3 is a flow diagram illustrating a process for determining, using contextual MAB models, user interaction policies based on contextual data, in accordance with certain embodiments of the disclosure. As described above, in order to obtain a more complete training dataset, including psychographic data, to train the contextual MAB models, the process 300 first performs a psychographic data collection and development phase 304 and then uses the more complete training dataset, including the collected and developed psychographic data, to train contextual MAB models that are used in an exploration-exploitation phase 306.

At block 302, in some embodiments, the process 300 includes performing a psychographic data collection phase. The data collection phase includes, a health monitoring platform (e.g., health monitoring platform 112) collecting psychographic data from users such as by providing users with a digital survey or quiz asking them to identify their goals, interests, and other types of psychographic data. In such an example, users may be provided with various user interfaces (“UIs”) on their mobile health applications that ask for psychographic data. For example, a UI may be presented to the user asking what the user's goal is and listing various goals (e.g., lower A1C, spend more time in range, feel better, lower stress, better sleep, more energy) that the user may select from. Other types of data (e.g., non-psychographic data) about the user may be collected as part of block 302 or separately. For example, the health monitoring platform may separately collect various other information about the users such as, but not limited to, device information, account information, demographics information, food consumption information, activity information (e.g., sleep and exercise), patient status information, medication (e.g., insulin) information, information from connected sensors such as blood glucose.

In some embodiments, the data collection phase (e.g., including psychographic data collection or collection of other types of data) may be continuous and ongoing or based on triggering events. For example, the psychographic data collection phase may be a progressive reveal where the user's mobile health application requests information from the user as the user uses the application. Other types of data, such as sensor data, may continuously be collected. In another example, the data collection phase may be triggered when the user's mobile health application determines that a confidence score for the data, e.g., psychographic data, that is available or inferred, is low. In such examples, the mobile health application may explicitly request user input.

As described above, not all users may respond to requests for psychographic data. For example, surveys, quizzes, or other types of request for psychographic data may be sent to 100,000 users. However, only 20,000 users may respond with responses to all psychographic related questions. As such, certain users may have incomplete psychographic profiles that are only partially complete or not complete at all. Therefore, to infer or predict psychographic data for the remaining 80,000 users, a plurality of imputation models may be trained, each configured to infer or predict a different psychographic feature, such as a goal, interest, behavior, concern, etc. Note that a psychographic profile is a type of contextual profile (e.g., a user profile comprising contextual information about the user, which may also include psychographic data).

At block 302, the process 300 includes training imputation models using a training dataset that includes the psychographic data, and/or other types of contextual data, collected during block 301. For example, a training dataset may be prepared using the data collected from the 20,000 users, the training dataset include all sorts of demographic, physiological, and other types of relevant data, as well as psychographic data. Using that training dataset, a plurality of imputation models may be trained, each configured to infer or predict a value for a different psychographic feature, such as a goal, interest, behavior, concern, etc. Depending on what an imputation model is configured to predict, the training dataset may be labeled differently. For example, if an imputation model is to be trained to predict a diabetic distress score, the training dataset is labeled with diabetic distress scores. The imputation models may be trained using one of various artificial intelligence/machine learning algorithms, such as supervised learning algorithms. In certain embodiments, the imputation models may be trained as classification, regression, or other types of models.

At block 303, the process 300 includes developing psychographic data, and/or other types of contextual data, for one or more users using the trained imputation models. Continuing with the example above, the trained imputation models may be used to develop psychographic profiles for the 80,000 remaining users whose psychographics profiles were incomplete. For example, an imputation model that is trained to predict a diabetic distress score for users is used to output predicted diabetic distress scores for the 80,000 remaining users. In another example, an imputation model that is trained to predict a hyperglycemia fear score for users is used to output predicted hyperglycemia fear scores for the 80,000 remaining users. In yet another example, an imputation model that is trained to predict the type of exercise users are interested in is used to output the type of exercise each of the 80,000 remaining users is interested in. Similarly, an imputation model that is trained to predict the type of glucose control goal users are interested in is used to output a predicted glucose control goal for each of the 80,000 remaining users.

The psychographic data that is imputed for the remaining 80,000 users is then used to build a training dataset that can be used to train the contextual MAB models utilized in block 306 for determining user interaction policies.

At block 306, the process 300 may include performing an exploration-exploitation phase for determining optimal user interaction policies. The exploration-exploitation phase may implemented using contextual MAB models and involve continuous exploration on some fraction of the user population to refine the contextual MAB models. The exploration-exploitation phase includes individualized or personalized contextual models to determine user interaction policies. In certain embodiments, the exploration-exploitation phase also includes utilizing observed outcomes to train and/or update the contextual MAB models and/or the imputation models discussed above. For example, the health monitoring platform may use feedback telemetry associated with the user interaction action policies provided to users to maximize user engagement and/or optimize health outcomes resulting from the user interaction policies.

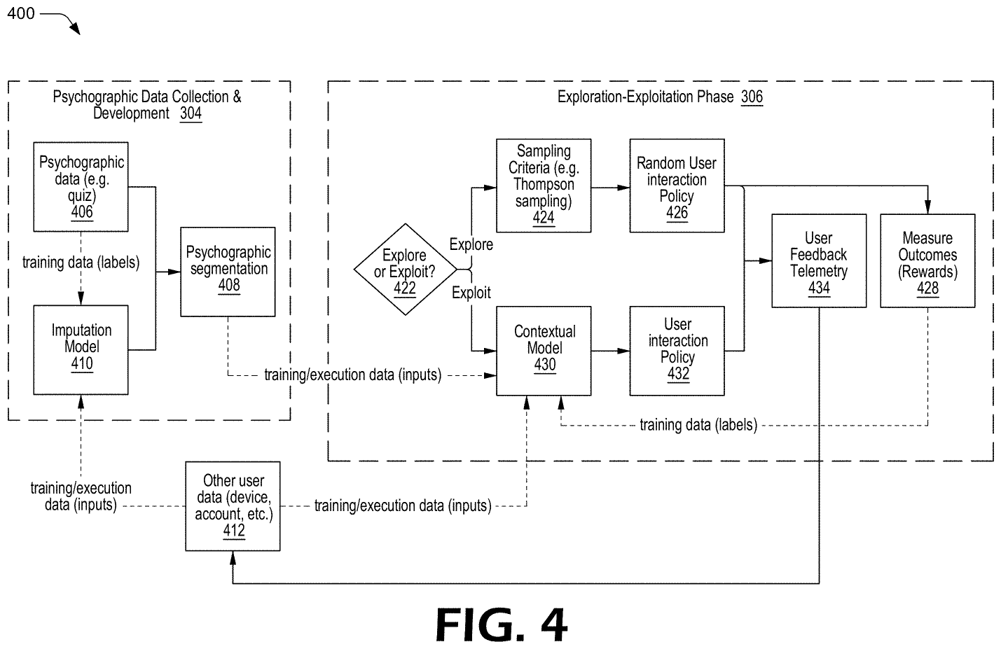

As described below in relation to FIG. 4, in the depicted exemplary exploration-exploitation phase 306, the health monitoring platform divides a set of users into an exploitation subset and an exploration subset. With respect to users in the exploitation subset, the health monitoring platform uses the contextual MAB models (e.g., a prediction model that uses contextual data) to determine user interaction policies based on the user's contextual data, including psychographic data. With respect to users in the exploration subset, the computing device randomly assigns user interaction policies and measures outcomes. In certain embodiments, the measured outcomes from the exploration subset of users may be used to explore potential user interaction policies that may be more advantageous and later assigned to the exploitation subset of users. Thus, the exploration-exploitation phase and the contextual MAB models may be utilized to determine optimal user interaction policies.

At block 308, the process 300 may also include the health monitoring platform retraining the contextual and/or imputation models. In some embodiments, the health monitoring platform may determine to continuously train the models. In some embodiments, the health monitoring platform may determine to retrain models in response to a trigger. For example, the health monitoring platform may determine to retrain periodically (e.g., every month, regardless of performance). In some embodiments, the process 300 may determine to retrain models every time the sample size of the exploration set of users' user interaction policies and observed outcomes hits a threshold number (e.g. 10,000). In certain embodiments, the health monitoring platform retrains the imputation and/or the contextual MAB models using user feedback telemetry and/or measured outcomes/rewards associated with the user interaction policies.

Below, blocks 304 and 306 are described in more detail and by reference to subsequent FIG. 4. In particular, block 304 of FIG. 3 is described in more detail by reference to blocks 406-412 of FIG. 4. Further, block 306 of FIG. 3 is described in more detail by reference to blocks 422-434 of FIG. 4.

### 1. Block 304: Psychographics Data Collection and Development Phase

As described above in reference to FIG. 3, process 300 may include performing a psychographic data collection and development phase 304. FIG. 4 is an exemplary operational process for performing the psychographics data collection and development phase in combination with the exploration-exploitation phase, in accordance with certain embodiments of the disclosure. The psychographics data collection and development phase may be performed to initially train imputation models or to retrain already-trained imputation models based on other user data (e.g., device, account, etc.) and/or user feedback telemetry. As described above, the imputation models are machine learning models that are configured to infer psychographic data (e.g., user's interests, goals, etc.) for a respective user when psychographic data is not available (or is not sufficiently available). The inferred psychographic data can be used generate psychographic profiles for users that can be used as part of a training dataset for training contextual MAB models used in the exploration-exploitation phase.

At block 406, psychographic data is collected for one or more users. For example, psychographic data may be collected using a quiz provided to each user, where the questions may be provided to a user via various UIs, as further described below. In some embodiments, each user's engagement with a UI may also provide psychographic data. For example, if the user engages with content (e.g., video, audio, text) that includes a particular subject matter, that subject matter may be used to collect psychographic data of the user, which can be indicative of the user's interest in such content. In certain embodiments, any metadata (e.g., user's search terms, time spent on a particular UI element, etc.) may be used to collect psychographic data of the user. As described above, psychographic data may be collected for a subset of the one or more users while remaining users may be unresponsive or provide incomplete information.

Once psychographic data is collected for the subset of users, at block 408, the psychographic data may be provided for constructing psychographic profiles (i.e., psychographic segmentations). For example, if a user has provided answers to a quiz, then the answers may be used for generating a psychographic profile including separating the user's psychographic data into one or more data entries that influence the user's behaviors. For example, a survey instrument (e.g. quiz) may include a series of questions that users can answer to determine a user's diabetes distress score. In another example, a survey instrument may be provided to a user regarding their fear of hyperglycemia or psychological well-being, etc. By issuing one or more surveys, if the data is available, then the answers for a user may be used to generate the training data set that then becomes input to subsequent contextual MAB models, as further described below.

For the remaining users for whom psychographic data is not available or is incomplete, the psychographic data collected for the subset of responsive users may be used in a training dataset to build imputation models. Thus, for users without psychographic data, the health monitoring platform may predict their psychographic data. For example, at block 410, the health monitoring platform may execute the one or more imputation models to infer the psychographic data for the remaining users. There may be an imputation model corresponding to each particular feature (e.g., diabetes distress, fear of hyperglycemia, psychological well-being, etc.) in the psychographic profile of the users. In other words, for each of these features (i.e., diabetes distress, fear of hyperglycemia, psychological well-being), there may be an imputation model for predicting a corresponding feature value. Each imputation model may be trained to take, as input, a user's demographic, physiological information, and other types of relevant information to predict a feature value of a certain psychographic feature in the user's psychographic profile.

In certain embodiments, the imputation models may infer psychographic data (and/or other types of contextual data) based on the best data available. If the inferred psychographic data is low confidence, or if there's an opportune time, the health monitoring platform may ask the user for that information to validate or retrain the imputation models on a regular basis. For example, for users that have indicated type 2 diabetes, the imputation models may infer that their goal is better sleep. The confidence level for the predicted goal of better sleep for this user, however, may be low. Therefore, the health monitoring platform may ask the user to validate whether sleeping better is a goal for the user. In some embodiments, even if the confidence level for the predicted goal of better sleep is high, the health monitoring platform may still confirm it with the user.

The imputation models may also be trained and retrained using user feedback telemetry from the exploration-exploitation phase 306, as shown in block 412. For example, after providing a user interaction policy (e.g., insight, recommendation, etc.) to a user, the user may be provided with a feedback element (e.g., a thumbs up, thumbs down, one through five star, etc.), and the user's feedback telemetry may be used to refine the imputation models. As an example, an imputation model may predict an interest for a user, such as running, and the exercise recommendations to the user may constantly indicate that the user should engage in running. However, the user may be interested in weightlifting instead. As such, user feedback telemetry may indicate that the user is not interested in running and, therefore used for retraining the imputation model.

### 2. Block 306: Performing the Exploration-Exploitation Phase

As described above in reference to FIG. 3, process 300 may include performing an exploration-exploitation phase using contextual MAB models. FIG. 4 is an exemplary operational process for performing the exploration-exploitation phase in combination with the psychographic data collection and development phase, in accordance with certain embodiments of the disclosure. The exploration-exploitation phase 306 may utilize contextual MAB models, at block 430, to optimize and assign user interaction policies as efficiently as possible. For example, the exploration-exploitation phase avoids having to “test” many different user interaction policies for individual users to see what works best. Instead, the exploration-exploitation phase involves training an algorithm to learn what the relationship is between user characteristics (e.g., psychographic data) and optimal user interaction policy (e.g., a recommended action) that result in optimal outcomes for the user. In certain embodiments, the exploration-exploitation phase determines user interaction policies in a manner that is intended to both: (i) maximize the likelihood that the user interaction policies lead to optimal outcomes (e.g., increase in user engagement, better health outcomes, etc.), and (ii) explore new user interaction policies while exploiting already-detected relationships between psychographic data (and/or other types of contextual data) and the user interaction policies.

At block 422, the exploration-exploitation phase 306 may include dividing a set of users into an exploration subset and an exploitation subset. As illustrated in block 424, the set of users are divided into an exploration subset and an exploitation subset using a sampling criteria. For example, the set of users may be divided where a ε fraction of the users are randomly selected and used to identify the exploration subset, and the remaining (1−ε) fraction of the users are used to develop an exploitation subset. In some embodiments, the magnitude of the exploration ratio ε decreases after each iteration of the exploration-exploitation phase. In some embodiments, the magnitude of the exploration ratio ε is set to zero after a predefined number of iterations of the exploration-exploitation phase. In certain embodiments, the magnitude of the decrease in exploration ratio ε may be a function of the user interaction policy accuracy (which may improve over time). In addition, the exploration ratio c may also have a lower bound >0, to ensure some degree of exploration is always performed since what works best for users may change over time.

For the exploration subset of users, at block 426, the health monitoring platform may randomly assign a user interaction policy (e.g., “random” action). At block 428, the health monitoring platform may measure outcomes that may be used to train/retrain the contextual MAB model at block 430. In some embodiments, user feedback, at block 434, may also be received and used to train/retrain the contextual MAB and/or imputation models at block 410.

For the exploitation subset of users, at block 430, the exploration-exploitation phase may include executing contextual models using contextual data (e.g., including psychographic data) for each user or user cohort to determine user interaction policies. For example, one of the many contextual models may be a decision support model that is a user-facing algorithm that determines user interaction policy, such as a decision support recommendation, for a user based on the user's contextual data.

At block 432, user interaction policies may be provided for each user of the exploitation subset of users. At block 434, the health monitoring platform may receive user feedback telemetry indicate of the effectiveness of the corresponding user interaction policy, for example, on user engagement, health, etc. For example, when the user interaction policy recommends a video, the user's engagement with the video may indicate positive user feedback telemetry. However, if the user does not engage with the video, then the user feedback telemetry may be determined to be negative. In another example, the recommend video may also include rating element (e.g., a thumbs up or thumbs down element) allowing the user to provide direct indication of positive or negative user feedback telemetry. In a further example, the user interaction policy may recommend the user to perform some action (e.g., walk 5,000 steps today). The user may perform the action thereby indicating a positive user feedback telemetry or the user may decline to perform the action thereby indicating a negative user feedback telemetry.

In some embodiments, after implementing a user interaction policy (e.g., watching the recommended video or performing a recommended action), outcomes (e.g., user metrics, such as physiological metrics (e.g., glucose trends)) may be observed at block 428 to measure if the user's metrics have improved or deteriorated. For example, if the user has been provided a recommendation, but the user's metrics have deteriorated, then the measured outcomes may indicate a low reward. In another example, if the user has been provided a recommendation, but the user's metrics have not improved or have not improved enough, then the measured outcomes may indicate a low reward. As described above, user feedback telemetry

Implementations and examples of CMAB models are further defined in U.S. application Ser. No. 17/931,531, which is incorporated herein by reference. In addition, implementations and examples of providing decision support recommendation using recommender models based on users' goals and interests are further defined in U.S. application Ser. No. 17/241919, which is incorporated herein by reference.

### Exemplary User Interfaces for Providing User Interaction Policies and Collecting Data

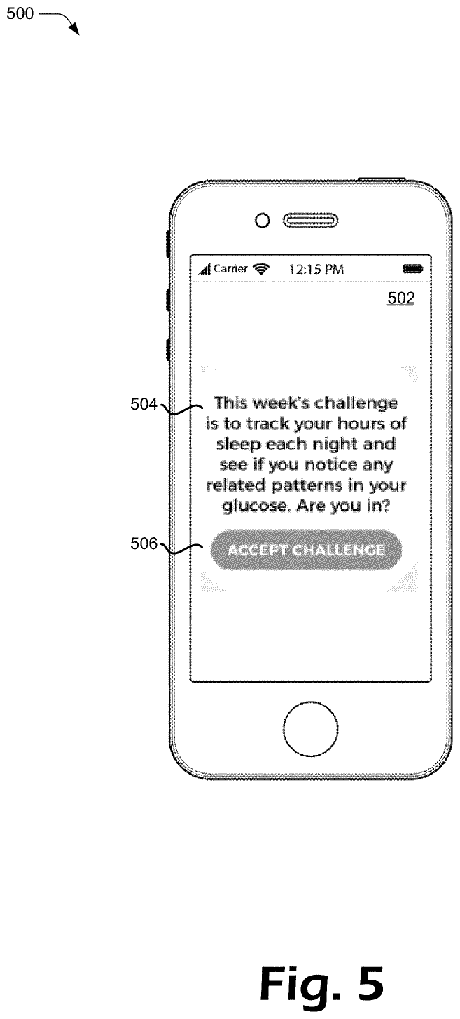

FIGS. 5-38 illustrate various UIs provided by a mobile health application executing on a display device (e.g., display device 108). Many of these UIs are used for providing user interaction policies to users. For example, some of the UIs are used to provide recommendations to users while other are used to provide insights or other information. In addition, user feedback telemetry may be received using the one or more UIs, as further described below.

Some other UIs may be used to collect data. As described further above, the present embodiments may include a data collection phase (and other data collection steps) to collect psychographic data and/or other types of data from users. Such data may be (1) used to train imputation models for inferring psychographic data (or other types of data) for users for whom psychographic data is not available (e.g., new users or users who were not responsive to data collection efforts) and (2) used as contextual data to provide optimal user interaction policies to users. Therefore, for example, a user may provide psychographic data directly via one or more UIs, as further described below.

The exemplary UIs illustrated in FIGS. 5-38 may include achievement UIs, family united UIs, journey together UIs, glucose inspiration UIs, follower highlights UIs, expert portal UIs, personalization UIs, appointment preparation UIs, video stories UI, and heath hub UIs. Each of these UIs may be used to gather psychographic data or other types of data, provide user interaction policies, and receive user feedback telemetry. For example, personalization UIs may present a quiz element that allows the user to provide psychographic data. Thus, some users may tell you what their goals are and what they wish to optimize. This psychographic data may be used as training data to build imputation models and to generate psychographic segmentations for training contextual models of the exploration-exploitation phase, as further described above. In this example, the collection of the psychographic data may be part of a progressive reveal. In some embodiments, it may be optional. In some embodiments, users may not provide answers to the quiz elements and thus the present embodiments may infer the psychographic data using the trained imputation models, and may try again to gather or confirm such data at a later stage in their progressive reveal. Thus, the present embodiments may allow for the health monitoring platform to impute psychographic data (e.g., psychographic attributes like goals and interests) before they are available.

### Achievement UIs

In implementations, such as those depicted in FIGS. 5-8, users earn badges for achieving milestones or completing challenges related to their diabetes management. In certain embodiments, the challenges may be selected based on the user's psychographic profile generated during the physchogrpahic data collection and development phase. The milestones and challenges range in difficulty—some are relatively easier than others to achieve. A mobile health application (e.g., analyte monitoring application) prompts a user with challenges related to exercise, healthy eating, stress management, glucose levels, and more. Every time the user completes a challenge or achieves a milestone based on their analyte data, the user can earn a unique badge that the application tracks on an achievements page of the mobile health application. Users can also choose to join a team with other users of the application and work together to complete team challenges, in addition to their own individual challenges.

FIG. 5 depicts an example of an achievement UI displayed in one or more implementations, in accordance with certain embodiments of the disclosure. In the example 500, user interface 502 includes a prompt element 504 with a challenge for the user. For example, the prompt element 504 states “This week's challenge is to track your hours of sleep each night and see if you notice any related patterns in your glucose. Are you in?” The user may then accept the challenge using a button element (i.e., “accept challenge” button) 506 indicating that the user will accept the stated challenge.

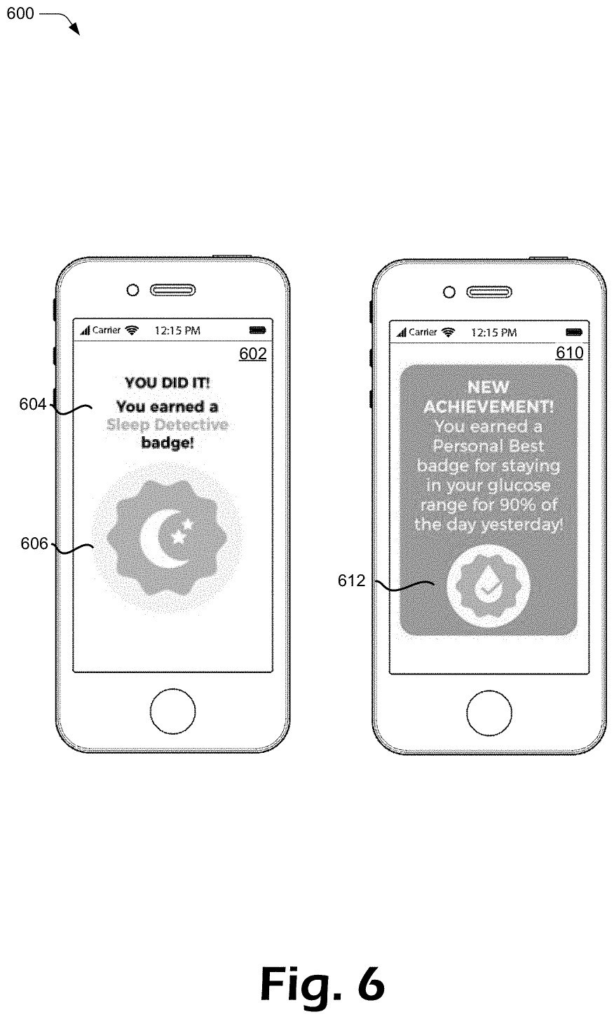

FIG. 6 depicts other examples of achievement UIs displayed, in accordance with certain embodiments of the disclosure. In the example 600, the achievement UI 602 includes a text element celebrating the user's achievement (i.e., “You did it! You earned a Sleep Detective badge!”) and includes a unique badge 606 earned by the user by completing a challenge. In addition, the achievement UI 610 includes a badge/reward 612 earned based on the user's analyte data. In example 600, the badge displayed in interface 610 corresponds to the user staying within a glucose range for a percentage of a day, e.g., staying between a low glucose threshold and a high glucose threshold for 90% of a day. Notably, the badges and awards may be based on metrics other than just analyte data. For example, the badge/reward 606 displayed in the achievement UI 602 is based on sleep data of the user.

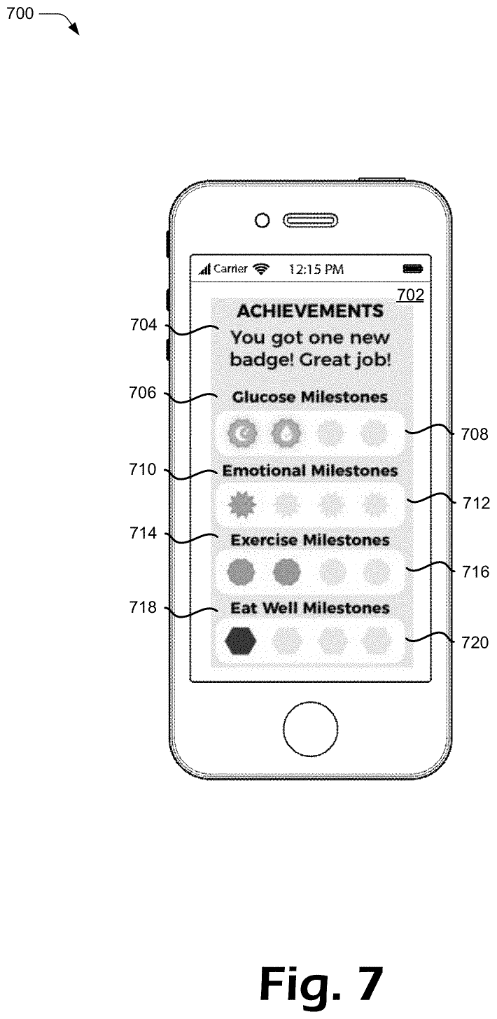

FIG. 7 depicts another example of an achievement UI displayed, in accordance with certain embodiments of the disclosure. In the example 700, the achievement UI 702 includes a text element, e.g., an “achievements” page and displays badges earned by a user. In example 700, the badges correspond to different milestones associated with different categories, such as glucose milestones 706 and badges 708, emotional milestones 710 and badge 712, exercise milestones 714 and badges 716, and nutrition milestones 718 and badge 720. Notably, a user may be able to earn multiple awards for each category by achieving various different milestones or completing challenges. A first glucose milestone 706, for example, may correspond to staying within a glucose range for 50% of a day, whereas a second glucose milestone may correspond to staying within the glucose range for 75% of a day.

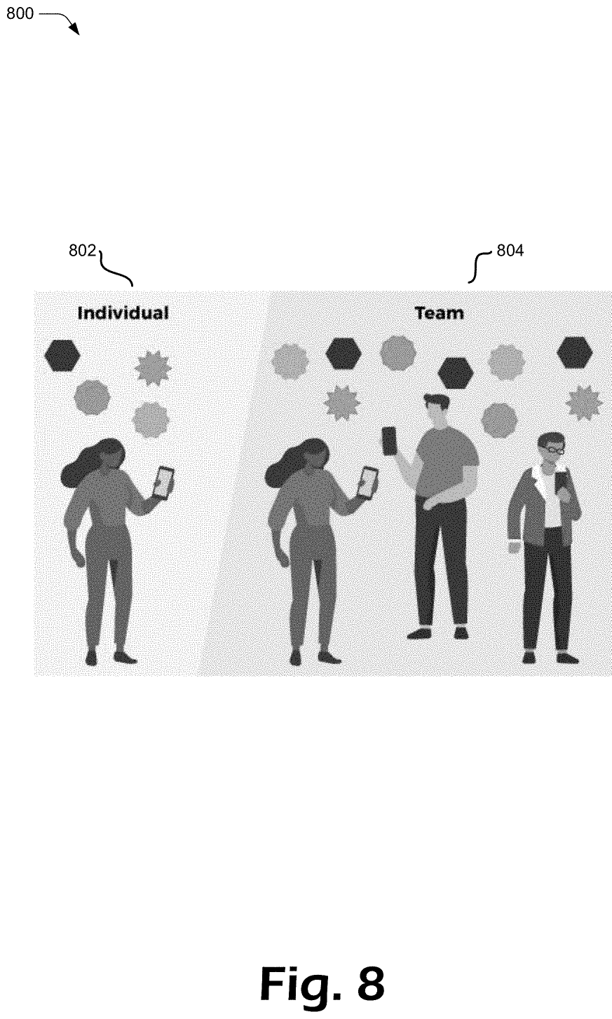

FIG. 8 depicts example badges displayed via achievement Uls of a user and via achievement Uls of a team of users that are competing together, in accordance with certain embodiments of the disclosure. In the example 800, badges are depicted for a user 802, e.g., based on individual challenges in which the user participates. In the example 800, badges are also depicted for a team of users 804, e.g., based on team challenges in which those users participate. The badges from the example 800 are displayed via respective achievement Uls.

### Family United UIs

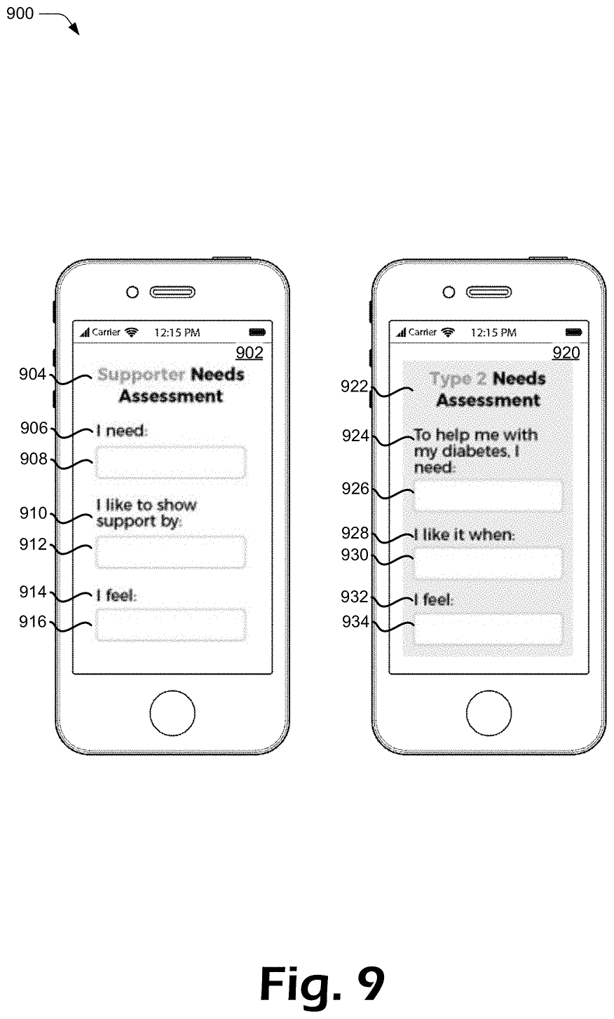

In implementations, such as those depicted in FIGS. 9-12, the mobile health application allows a group of users (e.g., a family, a group of friends, a sports team, etc.) associated with a particular user to interact in various ways that support the particular user. For example, the user's whole family can use the application together to better support the user. In one or more implementations, when a user first logs in to the application, the user may take a short quiz that gathers the user's psychographic data including the user's needs and preferences for how the user would like to be supported in health (e.g., diabetes) management. Each person in the user's group (e.g., in the user's family) will take a similar quiz about their preferences, thus providing psychographic data, when it comes to supporting the user. The application then generates recommendations on discussion points and educational suggestions to facilitate conversations around an adverse condition related to analyte monitoring, e.g., Type 2 diabetes. From there, the application allows users to send each other messages in appreciation of positive behaviors like buying diabetes-friendly groceries or going on a walk together.

FIG. 9 depicts an example of family united UIs displayed, in accordance with certain embodiments of the disclosure. In the example 900, the family united UI 902 depicts at least a portion of a quiz taken by a supporter (e.g., a family member) of the particular user. For example, the family united UI 902 includes a text element (i.e., “Supporter Needs Assessment”), a first question element 906 (i.e., “I need:”) and a first answer element 908 that allows the supporter to provide an answer to the first question element 906, a second question element 910 (i.e., “I like to show support by:”) and a second answer element 912 that allows the supporter to provide an answer to the second question element 910, and a third question element 914 (i.e., “I feel:”) and a third answer element 916 that allows the supporter to provide an answer to the third question element 914. Further, the family united UI 920 depicts at least a portion of a quiz taken by the particular user. For example, the family united UI 920 includes a text element (i.e., “Type 2 Needs Assessment”), a first question element 924 (i.e., “To help me with my diabetes, I need:”) and a first answer element 926 that allows the user to provide an answer to the first question element 924, a second question element 928 (i.e., “I like it when:”) and a second answer element 930 that allows the user to provide an answer to the second question element 928, and a third question element 932 (i.e., “I feel:”) and a third answer element 934 that allows the user to provide an answer to the third question element 932.

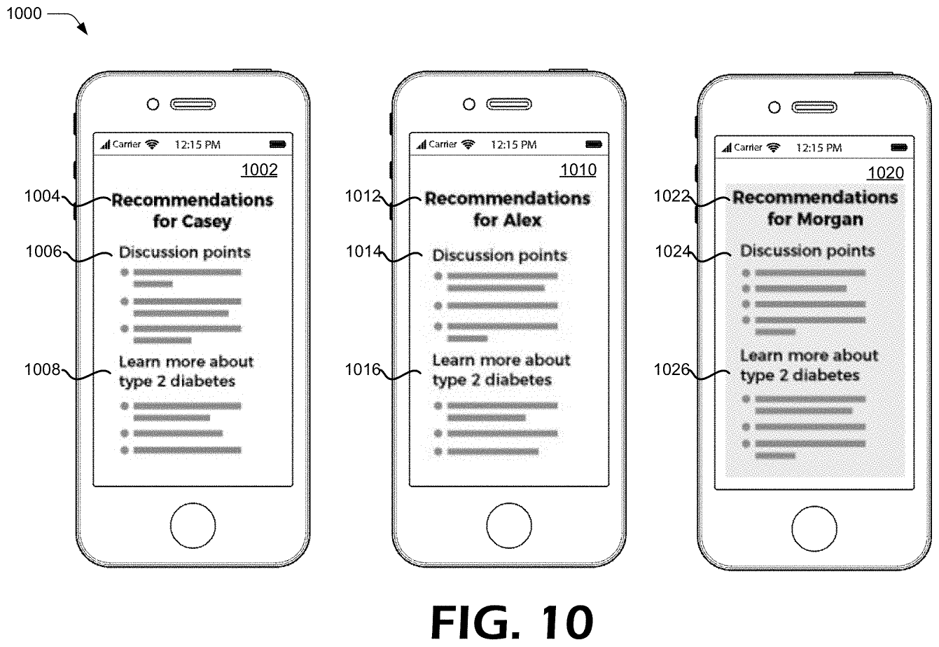

FIG. 10 depicts another example of family united UIs displayed, in accordance with certain embodiments of the disclosure. In the example 1000, the family united UIs 1002, 1010, 1020 depict personalized recommendations displayed and/or generated by the mobile health application to facilitate conversations around adverse conditions or ways to provide support related to the analyte monitoring, e.g., Type 2 diabetes. For example, family united UI 1002 includes personalized recommendations for Casey 1004 including discussion points 1006 and information about type 2 diabetes 1008. Further, family united UI 1010 includes personalized recommendations for Alex 1012 including discussion points 1014 and information about type 2 diabetes 1016. In addition, family united UI 1020 includes personalized recommendations for Morgan 1022 including discussion points 1024 and information about type 2 diabetes 1026.

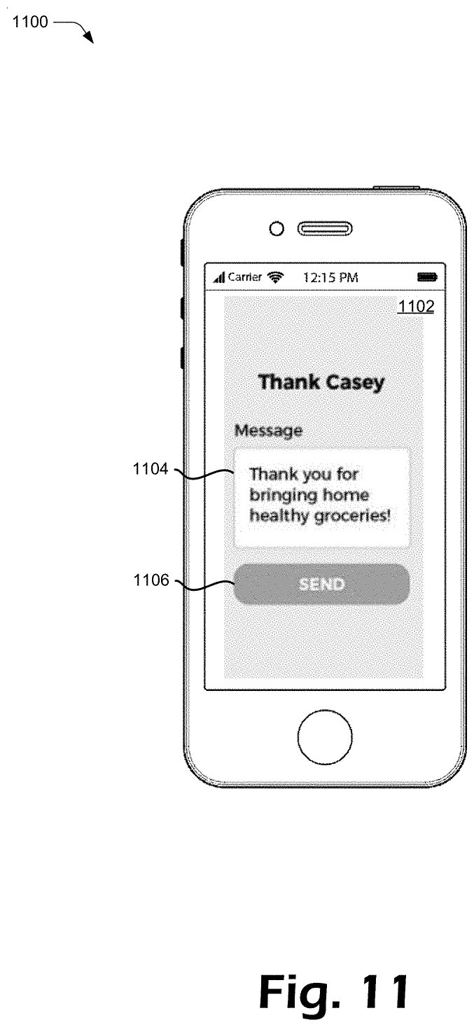

FIG. 11 depicts another example of a family united UI displayed, in accordance with certain embodiments of the disclosure. In the example 1100, the family united UI 1102 displays interface elements that enable users to send and receive messages in appreciation of positive or helpful behaviors. The family united UI 1102 includes a message box element 1104 that allows a participant in the group to send a message, using the button element 1106 (i.e., “send” button), to the user to thank the user for taking action to improve the management of diabetes (e.g., by thanking the user for bringing home healthy groceries).

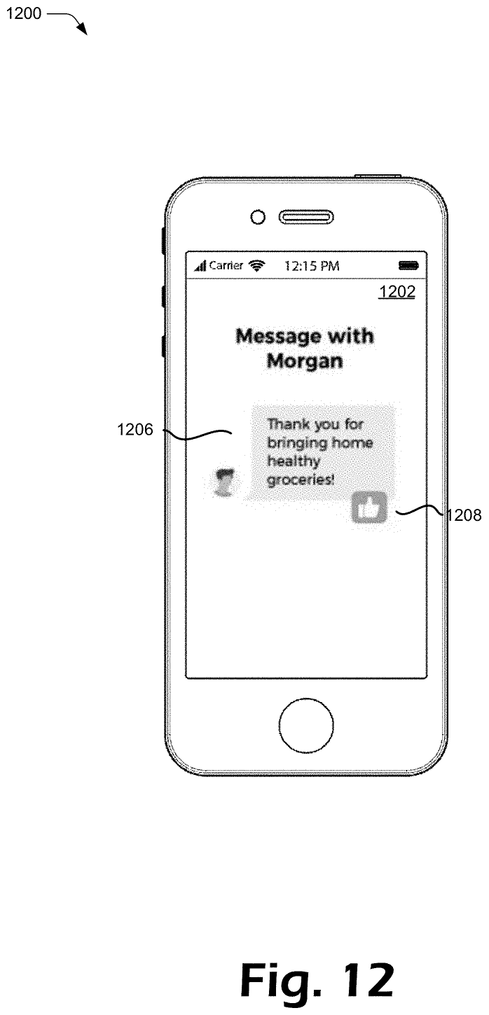

FIG. 12 depicts another example of a family united UI displayed in one or more implementations, in accordance with certain embodiments of the disclosure. In the example 1200, the family united UI 1202 displays interface elements that enable users to provide feedback indicating how they feel about messages received. For example, the family united UI 1202 to may display the received message 1206 and include a like button (i.e., the thumbs up button) that allows the recipient to provide feedback on received messages, such as those sent from another user via the family united UI 1102 depicted in the example 1100.

Additionally, the implementations depicted in FIGS. 9-12, enable users that have established accounts in association with a particular user, to let the particular user know that they are supported. The mobile health application can be used by a family together (or another group) to better support someone with an adverse health condition related to the analyte being monitored, e.g., Type 2 diabetes.

### Journey Together UIs

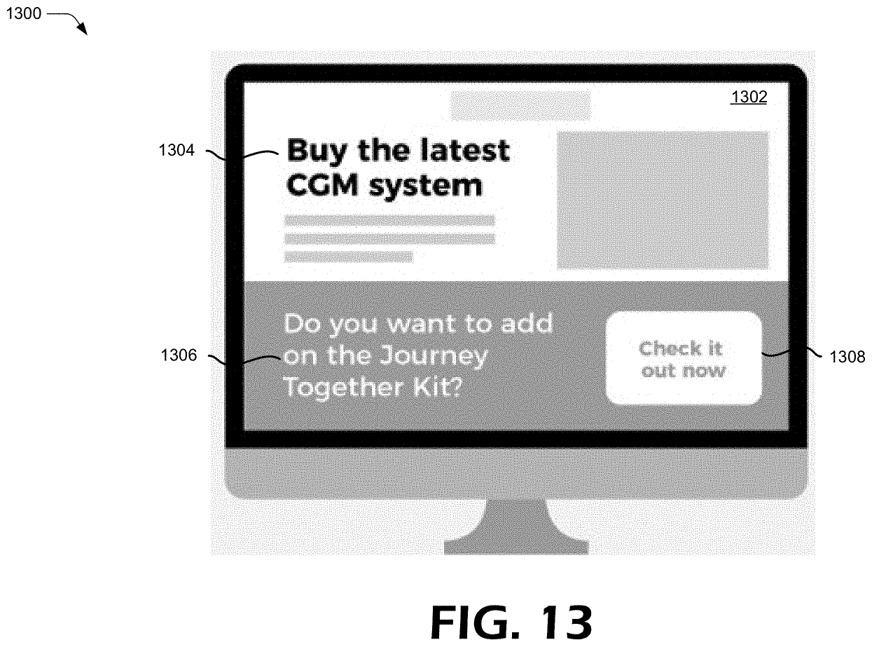

In implementations, such as those depicted in FIGS. 13-15, a particular user is able to share/start the particular user's analyte monitoring journey together with other users. When the user purchases an analyte monitoring device, the user is presented (e.g., displayed) the option to buy a kit that includes an analyte monitoring device for her-him-self, along with an extra analyte monitoring device(s) for one or more of the user's supporters to try for a period of time, e.g., one month. The user and the selected supporters can thus each wear the analyte monitoring device and learn about it together by sharing their data via the mobile health application. The application may thus gather psychographic data from the user and selected supporters and provide user interaction policies to optimize the user's health outcomes and the supporters' ability to provide support to the user. The application enables a group of associated users to share the experience of reviewing glucose data, whether or not they have diabetes. A user and their selected group can even do challenges as a team and to achieve the user's health goals together, e.g., through fun games. At the end of the month, the mobile health application sends one or more of the users a report with everyone's analyte data and, optionally, one or more educational insights based on analyte trends.

FIG. 13 depicts an example of a journey together UI displayed, in accordance with certain embodiments of the disclosure. In the example 1300, the journey together UI 1302 (displayed via a display device) includes a first text element 1304 that displays information about the CGM system and an option for a user to begin a “journey together” program with one or more other users, e.g., in which the user and the other users wear analyte monitoring devices for a period of time. For example, the journey together UI 1302 includes a second text element 1306 (i.e., “Do you want to add on the Journey Together Kit?”) and a button element 1308 (i.e., “Check it out now” button) that allows the user to begin the “journey together” program.

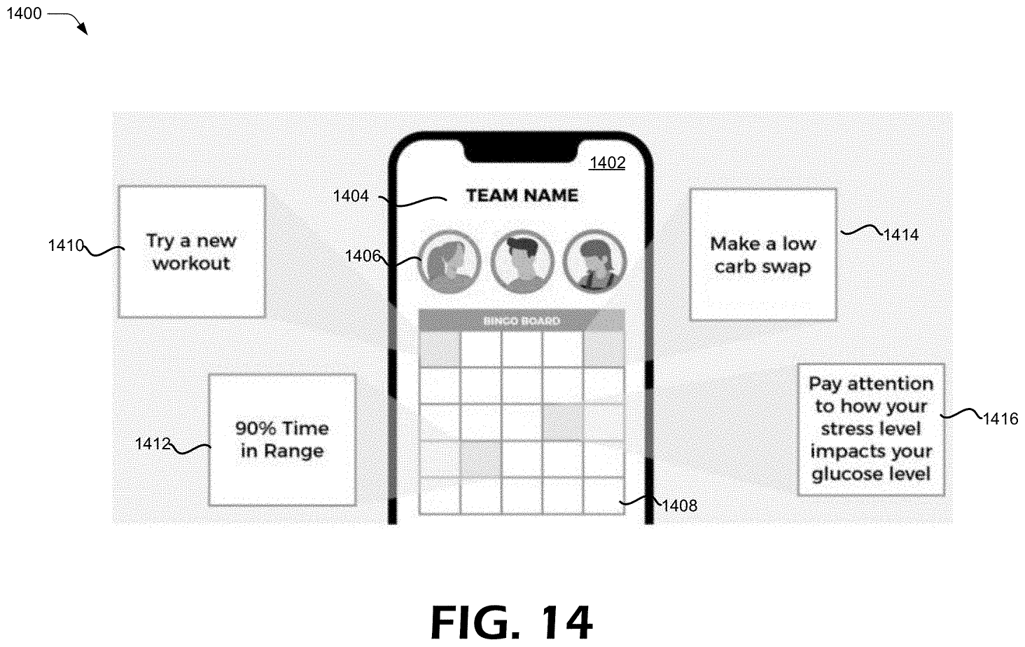

FIG. 14 depicts another example of a journey together UI displayed, in accordance with certain embodiments of the disclosure. In the example 1400, the journey together UI 1402 depicts a game used as part of the “journey together” program. The journey together UI 1402 includes a first text element 1404 identifying a group (i.e., “TEAM NAME”) and visual element 1406 showing the various users. Here, the game is displayed on a bingo board 1408, which includes analyte-related behavior boxes. The user can perform the various analyte-related behaviors (e.g., trying a new workout 1410, being within a glucose range for a particular percentage of a day 1412, swapping to a low carb diet 1414, or stress level management 1416) in order to “fill-in” related squares on the bingo board 1408.

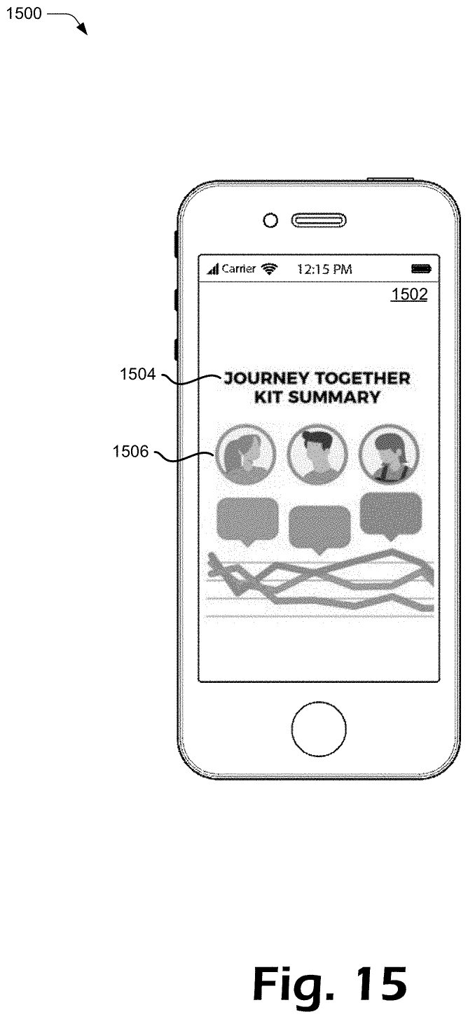

FIG. 15 depicts another example of a journey together UI displayed, in accordance with certain embodiments of the disclosure. In the example 1500, the journey together UI 1502 includes a first text element 1504 that displays a summary report of the group of associated users, e.g., the user's family, friends, or co-workers, selected via the journey together UI 1302 of FIG. 13. The journey together UI 1502 also includes a visual element 1506 showing the various users and analyte data (e.g., glucose traces) for all the users in the group and/or one or more educational insights for those users based on determined trends. In certain embodiments, the visual element 1506 may highlight each user's glucose data.

### Glucose Inspiration UIs

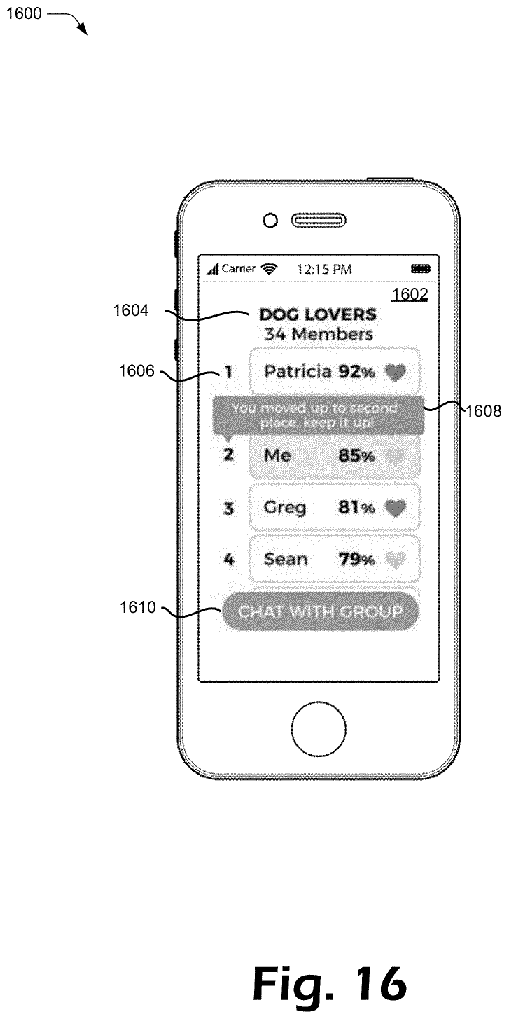

In implementations, such as those depicted in FIGS. 16-18, a mobile health application enables a user to compare how the user's glucose is doing at one or more points in time relative to other users on a similar journey. The user is presented with options to join groups of the user's choice which may be selected by the computing device based on the user's psychographic profile. Further, the application compares the metrics determined from the user's monitored analyte data (e.g., time in range) with metrics of other users, e.g., other users with Type 2 diabetes. The application enables users to send congratulations to others on a team selected as an option by the user. For example, if the user notices that another user is doing well, the user can send a congratulations message to encourage the other user to keep up the good work.

FIG. 16 depicts an example of a glucose inspiration UI displayed, in accordance with certain embodiments of the disclosure. In the example 1600, the glucose inspiration UI 1602 displays analyte-based metric data for users in a group which a particular user has selected to join and how group members' analyte-based metric data for a time period (e.g., a day) compares. For example, the glucose insipiration UI 1602 includes a first text element 1604 (i.e., “DOG LOVERS”) that identifies the group and the number of members (depicted as having 34 members). Further, the glucose inspiration UI 1602 also includes a ranking list element 1606, ranking the users based on an analyte-based metric data (e.g., percentage of time-in-range). In the depicted example, the ranking list element 1606 displays the user (i.e., “Me”) and a ranking text element 1608 indicating that the user has moving up to second place. The glucose inspiration UI 1602 may also include a button element 1610 that allows the user to chat with the group.

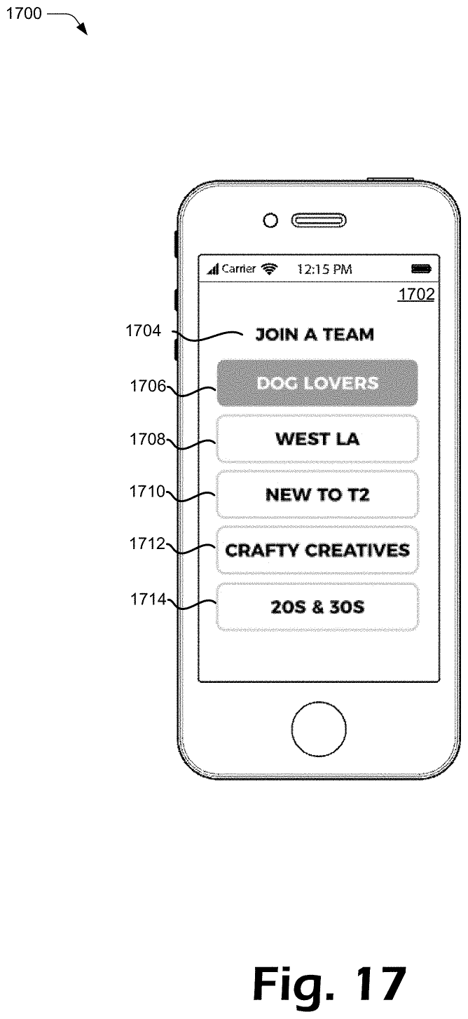

FIG. 17 depicts another example of a glucose inspiration UI displayed, in accordance with certain embodiments of the disclosure. In the example 1700, the glucose inspiration UI 1702 includes a text element 1704 (“JOIN A TEAM”) and button elements 1706-1714 that display selectable groups which a user can select to join, e.g., to compare the user's metrics with the metrics of other users in the group. Groups may be interest based, condition based, time based, age based, and location based, to name just a few.

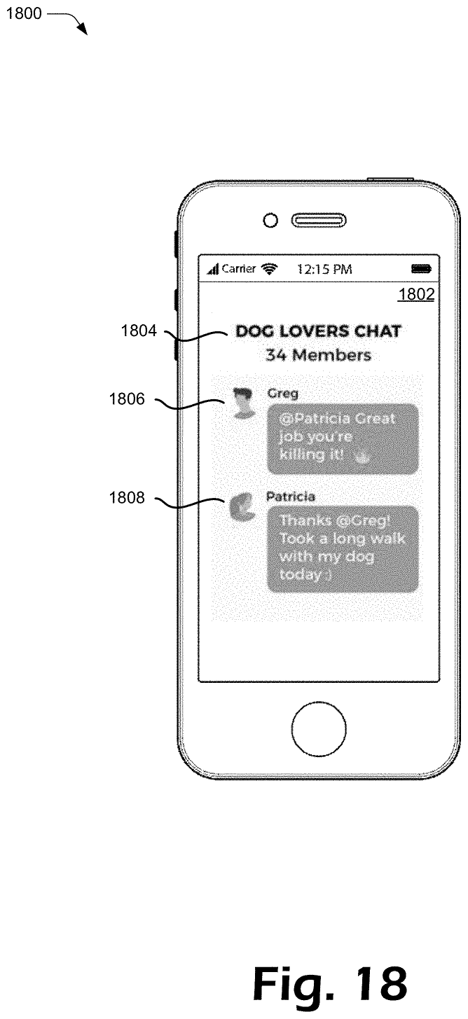

FIG. 18 depicts another example of a glucose inspiration UI displayed, in accordance with certain embodiments of the disclosure. In the example 1800, the glucose inspiration UI 1802 includes a first text element 1804 (i.e., “DOG LOVERS CHAT”) that identifies the group and the number of members (depicted as having 34 members). The glucose inspiration UI 1802 also includes messaging elements from members within the selected group (e.g., messege from Greg 1806 and message from Patricia 1808) that enable members of the group to send and receive messages with other members in the group.

### Follower Highlights UIs

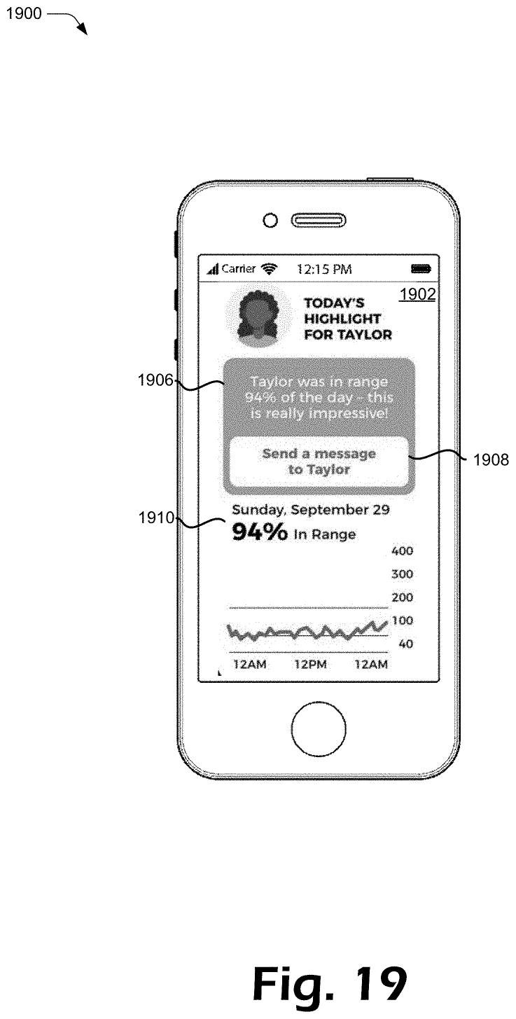

In implementations, such as those depicted in FIGS. 19-21, a mobile health application allows a user to receive recognition from family and friends based on analyte data and the user's psychographic data such as the user's goals and interests. A user can select to permit one or more supporter(s) to follow the user's analyte data, and permitted users may be sent or displayed daily highlights with explanations of what went well that day for the user. The one or more supporting users can also receive real-time notifications on their respective mobile health applications when the user is experiencing a good glucose moment. The mobile health application allows the supporting users to quickly send a message to the user, e.g., congratulating the user to show their support in the right moments. In certain embodiments, the messages may be selected based on the user's psychographic profile and what the contextual model determines is the optimal user interaction policy.

FIG. 19 depicts an example of a follower highlights UI displayed, in accordance with certain embodiments of the disclosure. In the example 1900, the follower highlights UI 1902 displays a user's analyte data (e.g., glucose data) and/or analyte highlights via a display device of a supporting user so that the supporting user can see what went well for the user. For example, the follower highlights UI 1902 includes a first highlight element 1906 that provides a highlight of a user's analyte data (i.e., “Taylor was in range 94% of the day—this is really impressive!”). The follower highlights UI 1902 also includes a button element 1908 that enables the supporting user to send the user a message. Further, the follower highlights UI 1902 also includes a second highlight element 1910 that provides details of the highlight of the user's analyte data such as, but not limited to, a glucose trend graph.

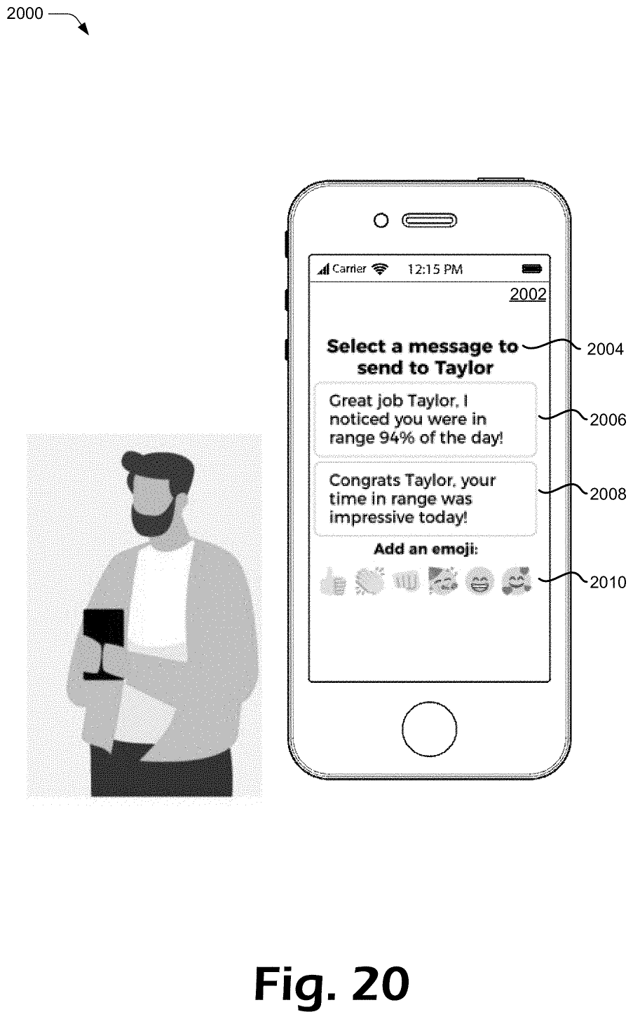

FIG. 20 depicts another example of a follower highlights UI displayed, in accordance with certain embodiments of the disclosure. In the example 2000, the follower highlights UI 2002 displays elements with which a supporting user can interact to send the user a quick message to the user to show support. For example, the follower highlights UI 2002 includes a prompt element 2004 (i.e., “Select a message to send to Taylor”) and a selection of quick responses including a first message 2006 (i.e., “Great job Taylor. I noticed you were in range 94% of the day!”), a second message 2008 (i.e., “Congrats Taylor, your time in range was impressive today!”), and a graphic element 2008 that includes a selection of graphics (e.g., emojis).

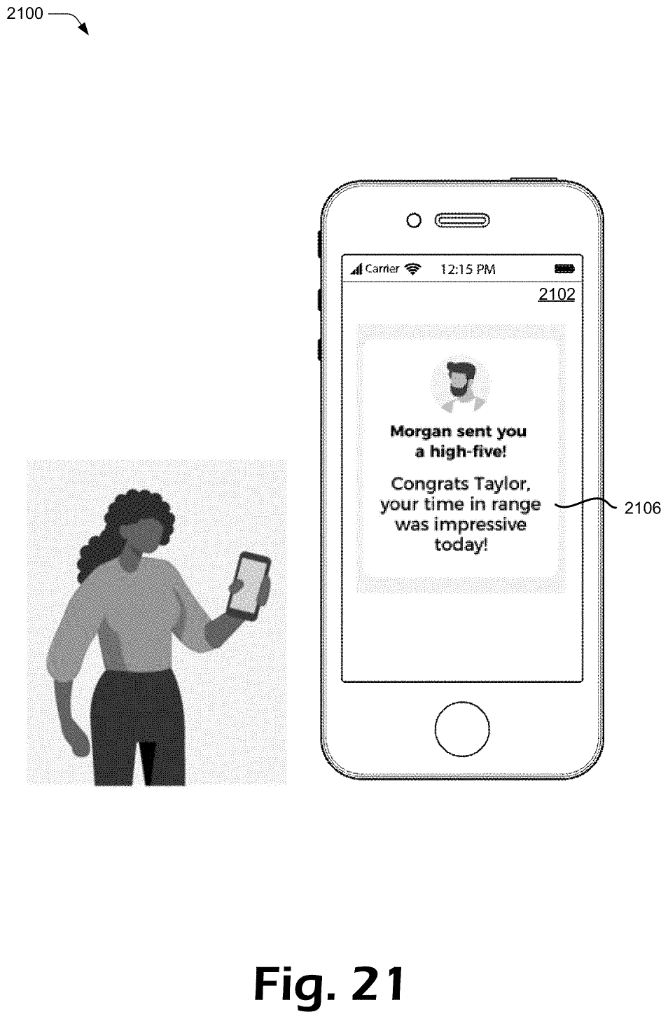

FIG. 21 depicts another example of a follower highlights UI displayed, in accordance with certain embodiments of the disclosure. In the example 2100, the follower highlights UI 2102 displays the message that the user receives from the supporting user. For example, follower highlights UI 2102 includes the message 2106 (i.e., “Congrats Taylor, your time in range was impressive today!”) that was sent by the supporting user.

In one or more implementations, the mobile health application enables a user to send other users, e.g., family and friends, praise based on their monitored analyte data. The mobile health application enables supporting users to follow a particular user's analyte data and to see daily highlights with explanations of what went well that day for the particular user. Supporting users can also receive real-time notifications a user the supporting user follows is experiencing a good glucose moment. The mobile health application allows supporting users to quickly send a message congratulating a followed user to show your support the followed user in the right moments.

### Expert Portal UIs

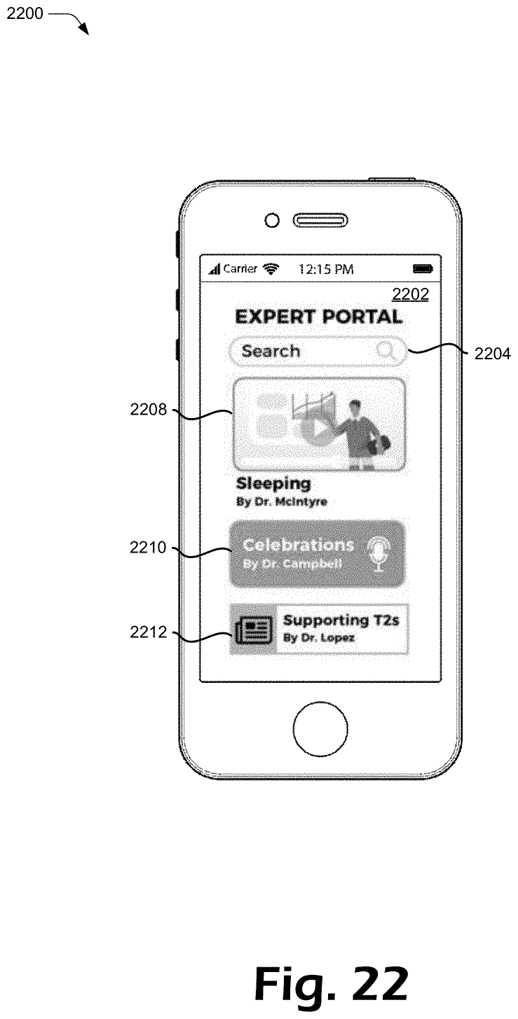

In implementations, such as those depicted in FIGS. 22-24, the mobile health application provides an expert portal. In some embodiments, the expert portal may be curated based on the user's interaction policy, as described above. Through the expert portal, a user is provided access to a content from a pool of diabetes experts supporting the user. In certain embodiments, the content may be personalized to the user's interest. The user is provided access to content on diabetes-related topics from healthcare professionals with different areas of expertise. The experts made available via the portal may include, by way of example and not limitation, doctors, diabetes educators, nutritionists, life coaches, and psychologists, to name just a few. In some embodiments, if a user has questions that are not yet addressed by the content in the portal's library, the user can submit a question and receive a response back from one of the experts in the portal. In some embodiments, the user can submit a question and receive content matches which may answer their question.

FIG. 22 depicts an example of an expert portal UI displayed, in accordance with certain embodiments of the disclosure. In the example 2200, the expert portal UI 2202 depicts elements that enable a user to search a library of content (e.g., videos, podcasts, and articles) from diabetes experts. The expert portal UI allows a user to watch, read, and/or listen to content on topics which may be pertinent to the user's search. For example, the expert portal UI 2202 includes a search element 2204 that enables the user to search the library of content. In such examples, the expert portal UI 2002 also provides results when search terms are provided by the user into the search element 2204. In other examples, the expert portal UI 2002 may preload content from the library of content before the user has activated the search element 2204. In both examples, the expert portal UI 2202 may provide link elements (e.g., video link 2208, audio link 2210, or text link 2212) to expert content.

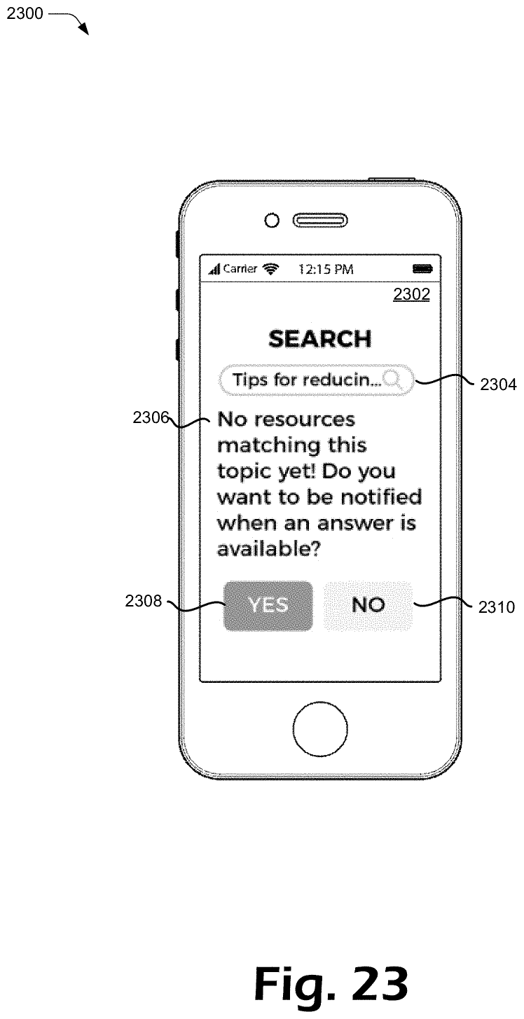

FIG. 23 depicts another example of an expert portal UI displayed, in accordance with certain embodiments of the disclosure. In the example 2300, the expert portal UI 2302 includes a search element 2204 that enables the user to search the library of content. When the library of content does not include the user's search, the expert portal UI 2302 includes a text element 2306 indicating that a match was not found and asking the user if they want to be notified when an answer is available. In such examples, the expert portal UI 2302 also includes a yes button 2308 and a no button 2310. When the yes button 2308 is indicated by the user, the user may be notified when a match is later found.

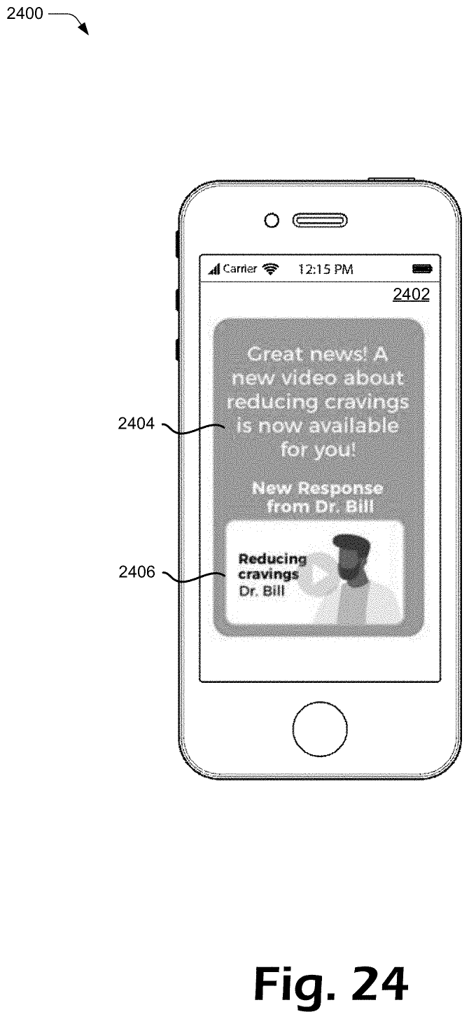

FIG. 24 depicts another example of an expert portal UI displayed, in accordance with certain embodiments of the disclosure. In the example 2400, the expert portal UI 2402 includes a notification element 2404 notifying a user when new content that may be relevant to the user has been found. For example, the user receives a response or is otherwise notified when a resource relevant to a submitted search or question is added to the portal. In the depicted example, the expert portal UI 2402 includes a video link 2406 to a video prepared by an expert. The video may be relevant to a submitted search or may answer a question submitted by the user. In one or more implementations, popular topics for a time period are presented via the expert portal UI 2402. For example, the topics that are the most asked-about by other users for the time period (e.g., a month) are presented via the expert portal UI 2402. In one or more implementations, the expert portal UI 2402 includes elements that are selectable by the user to upvote one or more of the popular topics, which further increases the popularity of an upvoted topic and increases the relative importance for an expert to answer.

### Personalization UIs

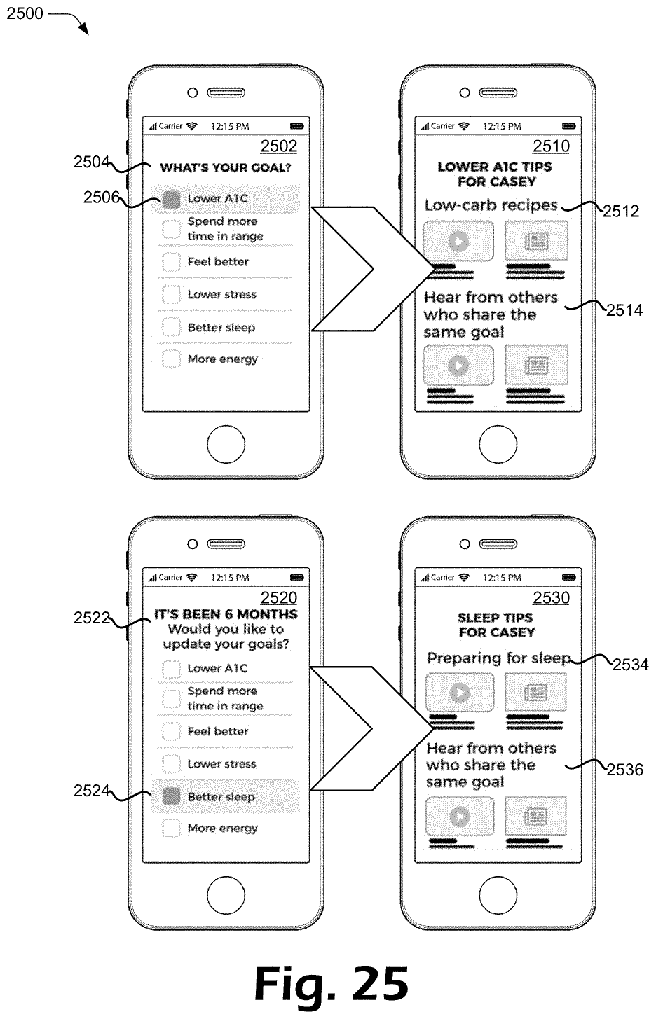

In implementations, such as those depicted in FIG. 25, the mobile health application enables a user to set and track personalized analyte-based goals. In this manner, the computing device may receive user psychographic data directly from the user. For example, the mobile health application enables a user to personalize his/her analyte monitoring experience based on the user's own individual goals. When the user first sets up a mobile health application, for instance, the user can be prompted to take a quick quiz to set health condition related goals (e.g., diabetes related goals). The tips and resources displayed to the user in the mobile health application are tailored to the user's needs and unique to the user. Since the user's priorities may change over time, the application enables the user to adjust this in the application's settings at any point

FIG. 25 depicts an example of personalization UIs displayed, in accordance with certain embodiments of the disclosure. In the example 2500, the personalization UI 2502 includes a quiz element 2504 that enables a user to take a quiz to set one or more health condition (e.g., diabetes) related goals. In certain embodiments, the quiz may be presented at various times, e.g., at initialization. Alternatively or additionally, the user may initiate a quiz, e.g., by navigating to the application's settings or some other interface. The personalization UI 2502, for example, lists a variety of different goals which can be selected by the user via one or more selection elements (e.g., selection element 2506 for “Lower A1C”), spend more time in range, feel better, lower stress, better sleep, more energy, and so forth.

After selection, the user may be provided with personalization UI interface 2510 that displays decision support outputs (e.g., tips and resources) curated for the user based on the user's selected goal as well other data associated with the user's goal. For example, personalization UI 2510 includes a first decision support output 25122 (i.e., “Low-carb recipes”) and a second decision support output 2514 (i.e., “Hear from others who share the same goal”). In certain embodiments, the tips and resources may be curated based on other user data. In one or more implementations, the mobile health application acknowledges when the user achieves a goal, e.g., by presenting a congratulatory message via the user interface.

Personalization Uls may enable a user to update and/or provide additional data related to their goals. For example, personalization UI 2520 displays elements that enable a user to take a subsequent quiz at a subsequent time, where the subsequent quiz enables the user selection one or more health condition related goals. In one or more implementations, the application prompts the user to take the subsequent quiz. The personalization UI 2520 includes a quiz element 2522 that enables the user to take a quiz to update one or more health condition (e.g., diabetes) related goals. In the depicted example, the user use a selection element 2524 to select “Better sleep” as an updated goal.

After selection, the user may be provided with personalization UI interface 2530 that displays decision support outputs (e.g., tips and resources) curated for the user based on the user's updated goal, data associated with the user's goal, or other user data. For example, personalization UI 2530 includes a first decision support output 2534 (i.e., “Preparing for sleep”) and a second decision support output 2536 (i.e., “Hear from others who share the same goal”).

### Appointment Prep UIs

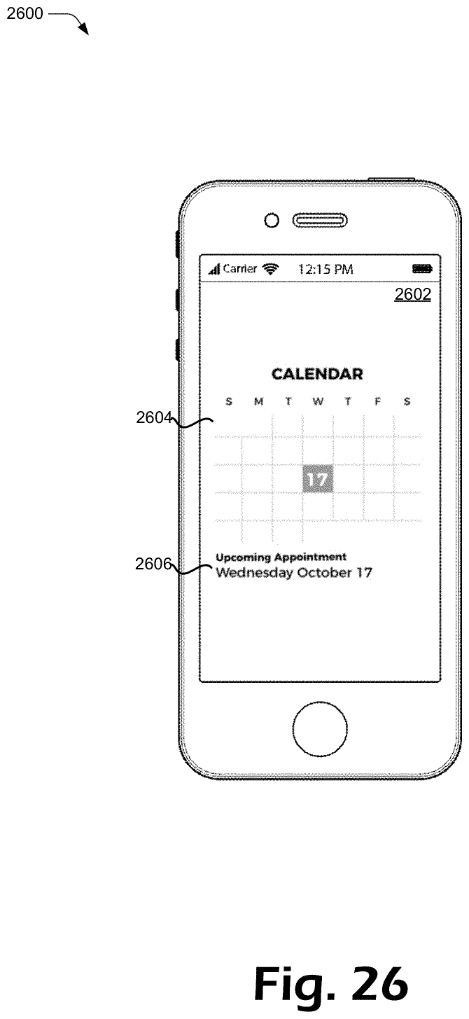

In implementations, such as those depicted in FIGS. 26-31, the mobile health application enables a user to prepare for healthcare provider appointments. An advantage of the application is that a user is able to better prepare for a healthcare provider's appointments. In the application, user interfaces display a user's upcoming appointments. Before each appointment, the mobile health application generates and provides a list of questions to ask a healthcare provider, which are editable by the user to fit the user's needs. In certain embodiments, the list of questions may be based on the user interaction policy. In addition, based on the user's analyte data and trends, the mobile health application may suggest questions to add to a running list. In addition or alternatively, the user can flag a day or moment (e.g. a particular glucose spike or pattern (e.g. hypoglycemia at time)) of interest in the analyte data and add notes to discuss that day's analyte data with the healthcare provider. Before the user goes to each appointment, the mobile health application provides a checklist with reminders of what to do in advance to make the appointment as efficient and valuable as possible. Users can store key takeaways/answers to questions after the appointment so that they can refer back to them at any point

FIG. 26 depicts an example of an appointment prep UI displayed, in accordance with certain embodiments of the disclosure. In the example 2600, the appointment prep UI 2602 includes a calendar element 2604 and text element 2606 that displays upcoming appointments and reminder notices, e.g., with a healthcare provider.

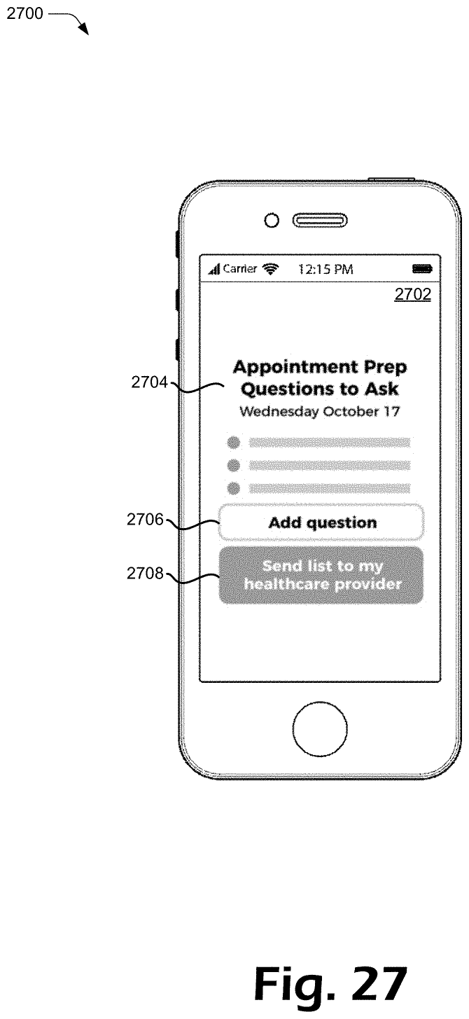

FIG. 27 depicts another example of an appointment prep UI displayed, in accordance with certain embodiments of the disclosure. In the example 2700, the appointment prep UI 2702 includes a list element 2704 that includes appointment prep questions to ask by the user at the appointment. The appointment prep UI 2702 also includes an add button element (i.e., “Add question”) that allows the user to add questions to the list of questions associated with an upcoming appointment. The appointment prep UI 2702 also includes a transmit button element 2708 that is selectable by the user to send the list of questions and the user's analyte data to a healthcare provider associated with the user. The application builds a list of questions for a user between appointments for the user to ask her healthcare professional. The list of questions may include application-determined questions and/or user-selected questions.

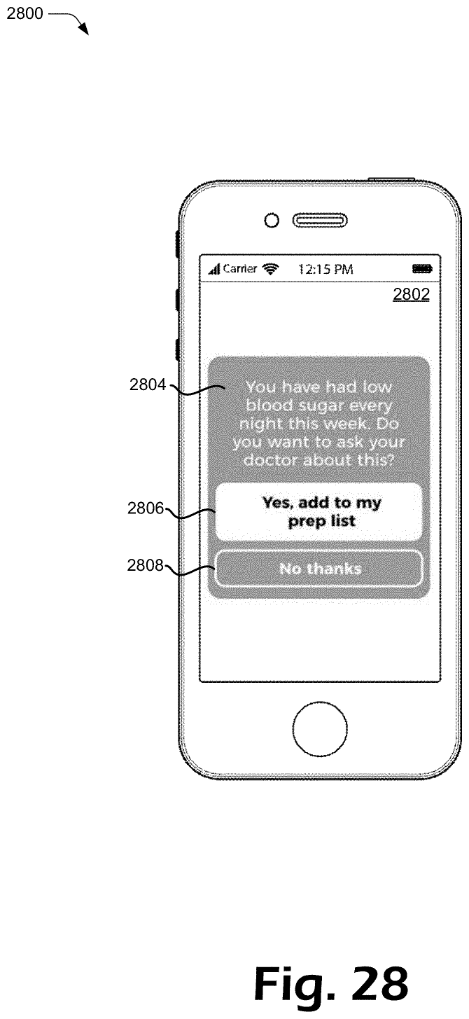

FIG. 28 depicts another example of an appointment prep UI displayed, in accordance with certain embodiments of the disclosure. In the example 2800, the appointment prep UI 2802 includes elements for adding a question to a user's list, where the user is prompted to add the question by the application based on an analysis of the user's analyte data over a period of time. For example, based on the user's glucose data, the system can determine that the user had low blood sugar every night during the previous week. Based on this determination, the system asks the user if the user would like to ask their doctor about their pattern of nighttime low blood sugar. In the depicted example, the appointment prep UI 2802 includes a prompt element 2804 indicating the user's analyte data (i.e., “You have had low blood sugar every night this week.”) and asking the user to add (i.e., “Do you want to ask your doctor about this?”). The appointment prep UI 2802 also includes an add button element 2806 (i.e., a button element that states, “Yes, add to my prep list”) and a decline button element 2808 (i.e., a button element that states, “No thanks”).

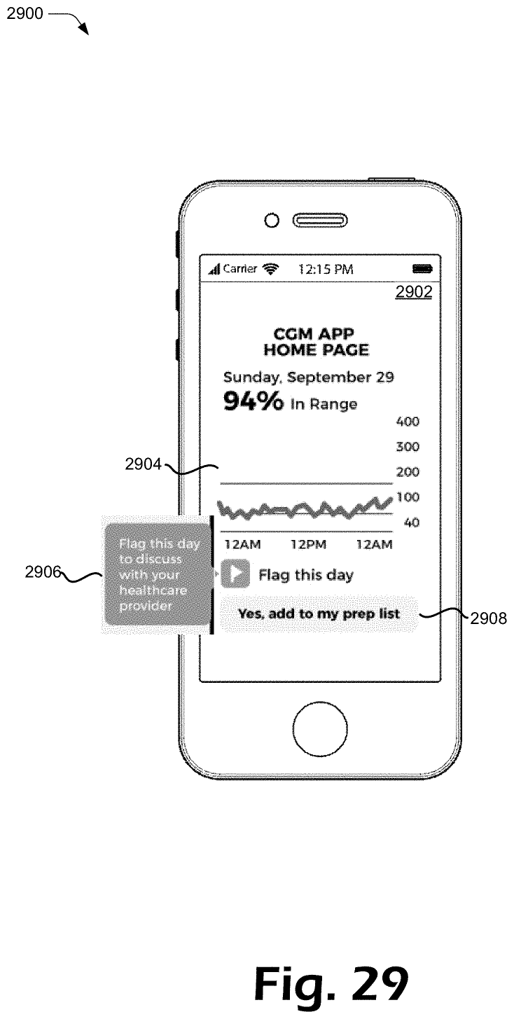

FIG. 29 depicts another example of an appointment prep UI displayed, in accordance with certain embodiments of the disclosure. In the example 2900, the appointment prep UI 2902 includes a trend graph element 2904 (e.g., indicative of the user's analyte concentration over a period of time) and a flagging element 2906, which allows the user to flag the period of time (e.g., day), which may cause the application to remind the user to discuss the analyte data for that time period with the user's healthcare professional. The appointment prep UI 2902 also includes an add button element 2908 (i.e., a button element that states, “Yes, add to my prep list”).

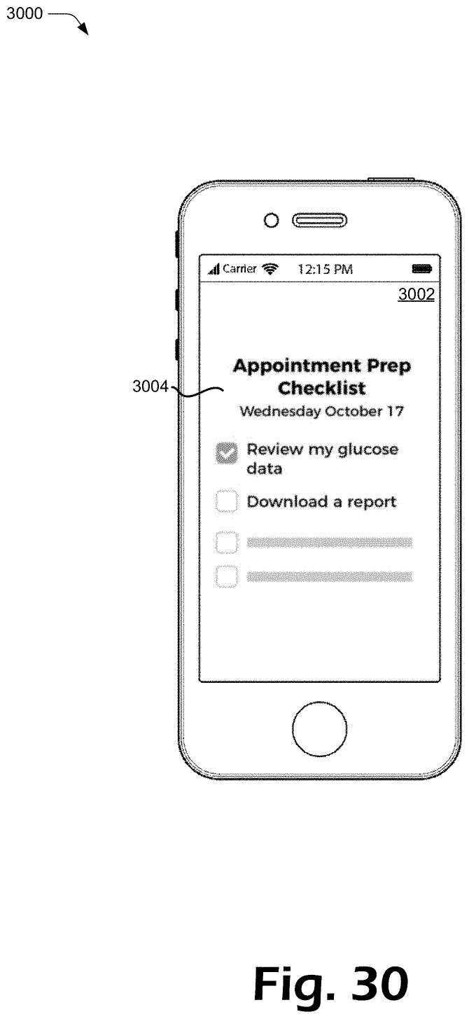

FIG. 30 depicts another example of an appointment prep UI displayed, in accordance with certain embodiments of the disclosure. In the example 3000, the appointment prep UI 3002 displays a checklist element 3004 that provides the user with a checklist (i.e., “Review my glucose data,” “Download a report,” etc.) that is reviewable by the user prior to an appointment with a healthcare provider, e.g., so the user and the healthcare provider can make the most out of the appointment.

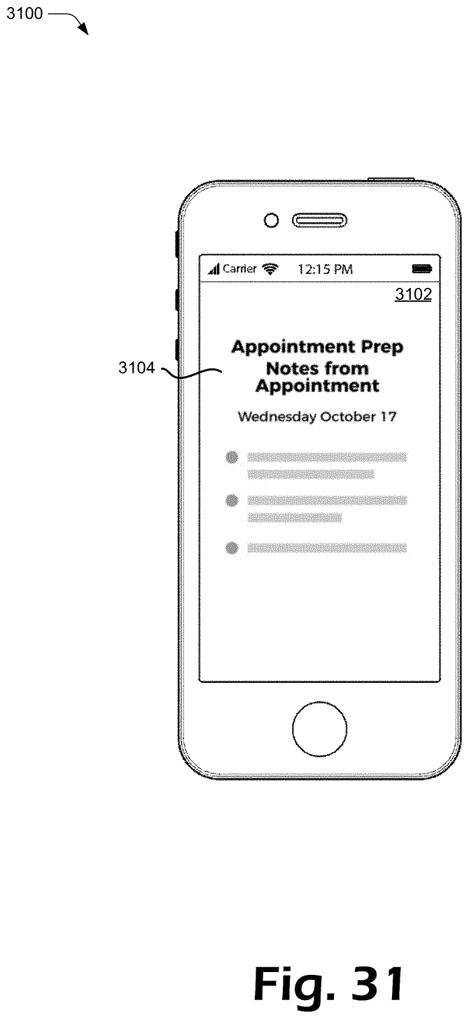

FIG. 31 depicts another example of an appointment prep UI displayed, in accordance with certain embodiments of the disclosure. In the example 3100, the appointment prep UI 3102 includes a notes list element 3104 that enables the user to document key takeaways/answers to questions after an appointment.

### Video Stories UIs

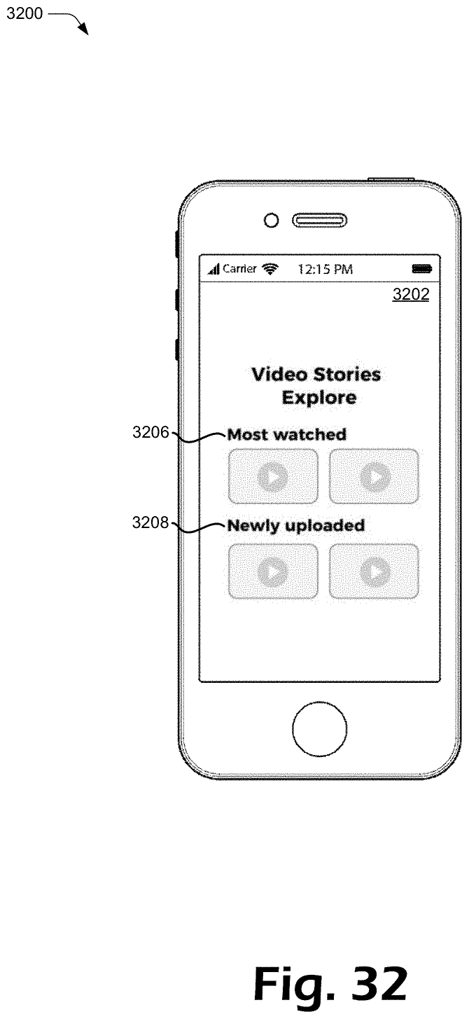

In implementations, such as those depicted in FIGS. 32-35, the mobile health application recommends digital content, e.g., videos based on the user's psychographic profile. The mobile health application enables a user to listen to and/or watch stories about other people with a health condition, e.g., Type 2 diabetes. When the user signs into the mobile health application and/or a corresponding website, a user interface presents high-quality videos (or other digital content) curated to the user based on various factors, including analyte data over a period of time (e.g., what the user's glucose looks like during the week) and other user data (e.g., demographics, historical selection of digital content, similarities with other users, activity data, meal data, medicament data, and so forth). The application also enables the user to interact with elements to filter the videos based on categories in which the user is interested. Additionally, the application enables the user to provide feedback in relation to the videos watched, and the next videos recommended by the application are tailored based on the feedback.

FIG. 32 depicts an example of a video stories UI displayed, in accordance with certain embodiments of the disclosure. In the example 3200, the video stories UI 3202 displays elements which enable a user to browse a collection of digital content. For example, the video stories UI 3202 includes video links organized based on different categories of videos (i.e., “Most watched” 3206 and “Newly updated” 3208).

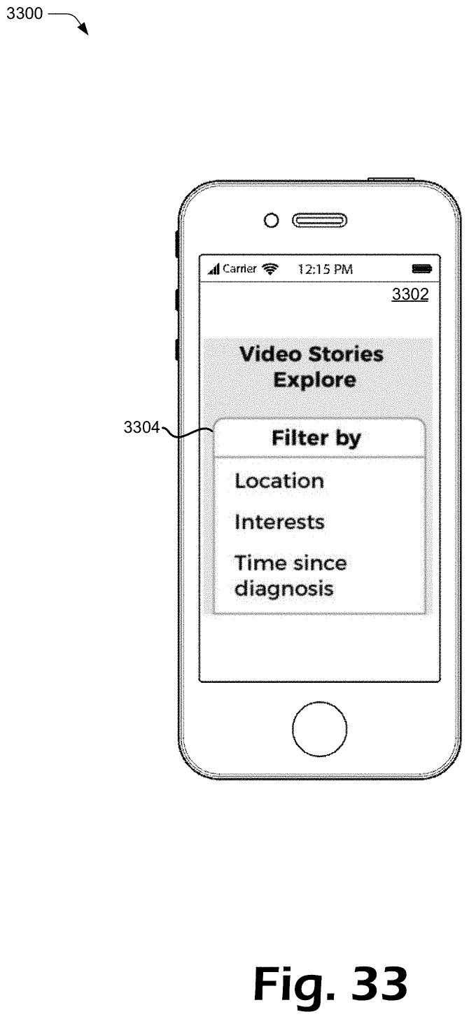

FIG. 33 depicts another example of a video stories UI displayed, in accordance with certain embodiments of the disclosure. In the example 3300, the video stories UI 3302 displays elements which are selectable to filter digital content based on the type of content the user is interested in consuming. For example, the video stories UI 3302 has a filter button 3304 that allows the user to filter digital content based on “Location,” “Interests,” “Time since diagnosis,” etc.

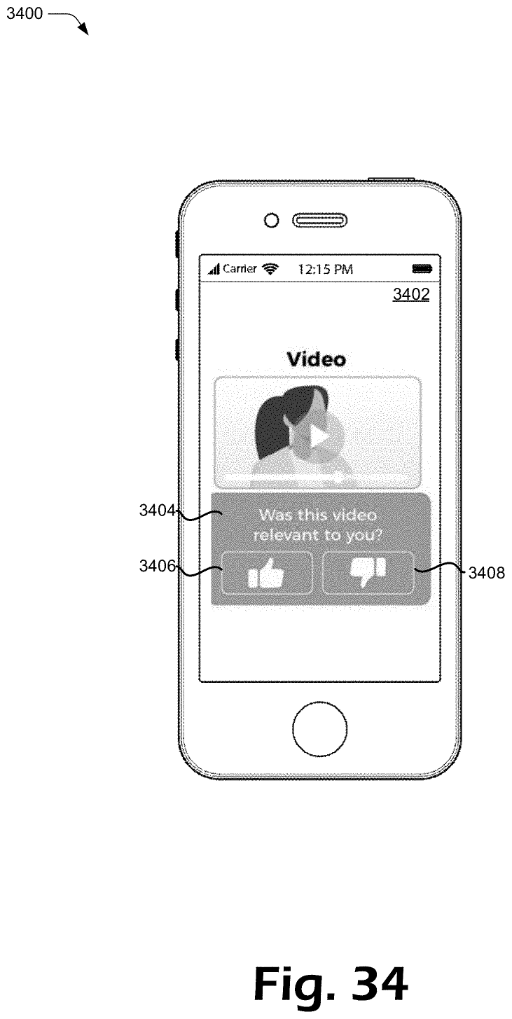

FIG. 34 depicts another example of a video stories UI displayed, in accordance with certain embodiments of the disclosure. In the example 3400, the video stories UI 3402 includes a feedback element which enables a user to provide feedback in relation to digital content output via the computing device, such as displayed via the user interface 3402. For example, the video stories UI 3402 may include a prompt element 3404 (i.e., “Was this video relevant to you?”) and a thumbs up element 3406 and a thumbs down element 3408.

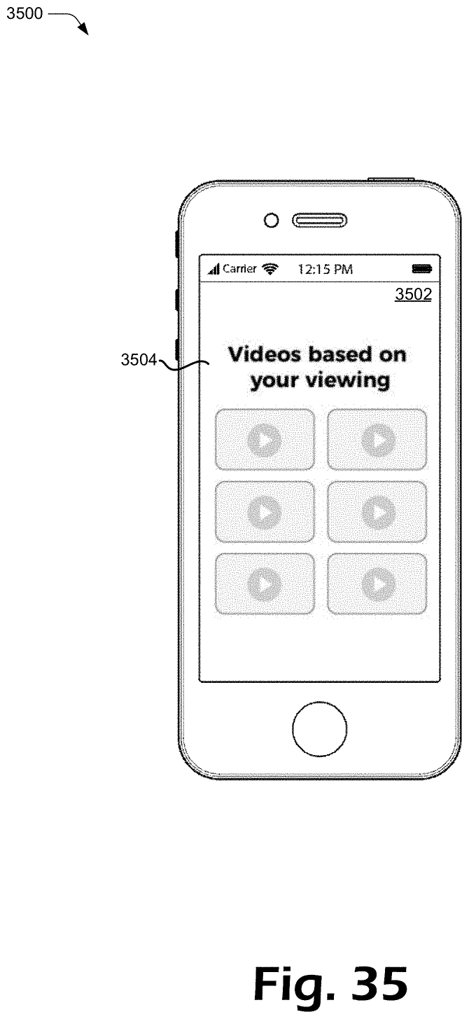

FIG. 35 depicts another example of a video stories UI displayed, in accordance with certain embodiments of the disclosure. In the example 3500, the video stories UI 3502 includes a suggestion element 3504 that displays recommended digital content tailored to the user's preferences, e.g., based on the user's feedback or based on the user's other watched videos.

In one or more implementations, the mobile health application outputs notifications when it is determined that at least one item of the digital content might provide helpful insight to the user, e.g., based on the analyte data of the user over a time period and/or based on metrics (e.g., trends) derived from the analyte data over time.

### Health Hub UIs

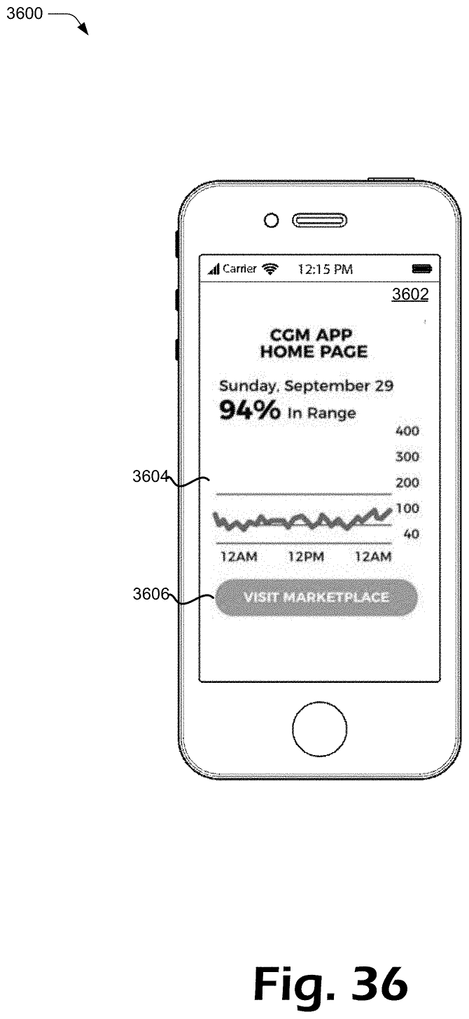

In implementations, such as those depicted in FIGS. 36-38, the mobile health application interfaces with a marketplace that enables interaction with additional third-party applications, which may be related to the mobile health application and based on the user's psychographic profile. The mobile health application connects with other applications, e.g., health related applications for a holistic experience. In the mobile health application, a user is able to choose other applications the user would like the mobile health application to connect with and to see data from. Based on user selections, the mobile health application will have access to high-level metrics from each application selected, all in one central location.

FIG. 36 depicts an example of a health hub UI displayed, in accordance with certain embodiments of the disclosure. In the example 3600, the health hub UI 3602 includes a trend graph element 3604 (e.g., graph plotting a trend of an analyte and/or an analyte-based metric over time) and a selection button element 3606 that is user-selectable for interacting with one or more other applications. In other words, the health hub UI 3602 allows the user access to a marketplace of other connected applications from a home page (or other page) of the mobile health application.

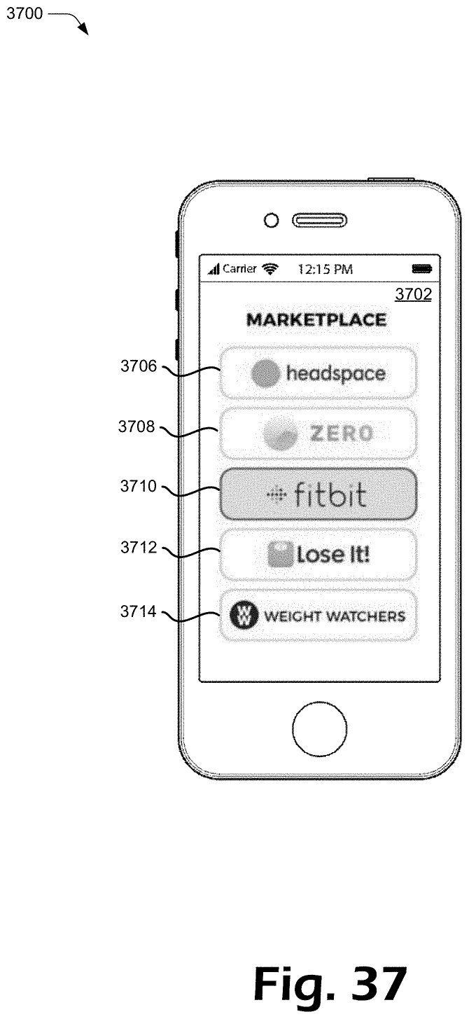

FIG. 37 depicts another example of a health hub UI displayed, in accordance with certain embodiments of the disclosure. In the example 3700, the health hub UI 3702 displays a plurality other application which are selectable to connect with the mobile health application. For example, the health hub UI 3702 includes a first button element (i.e., “headspace”), a second button element (i.e., “zero”), a third button element (i.e., “fitbit”), a fourth button element (i.e., “Lose it!”), and a fifth button element (i.e., “weight watchers”).

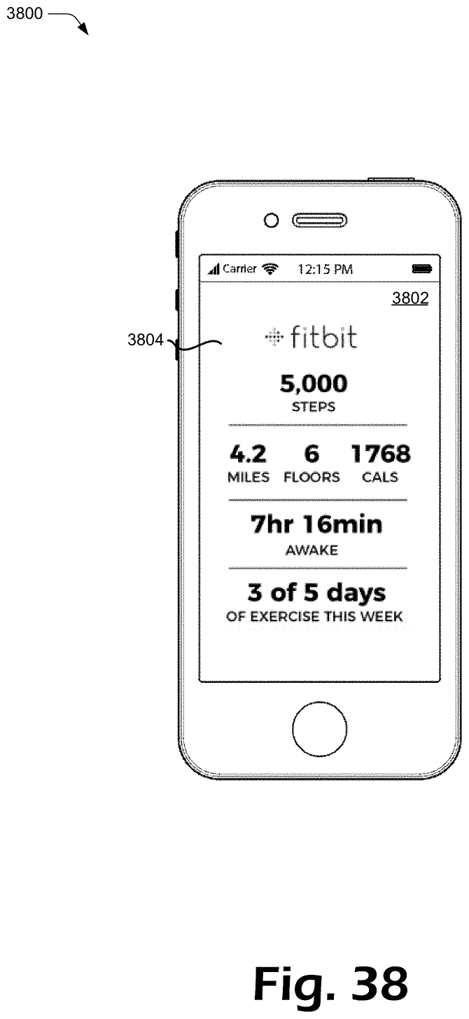

FIG. 38 depicts another example of a health hub UI displayed, in accordance with certain embodiments of the disclosure. In the example 3800, the health hub UI 3802 includes a presentation element 3804 that depicts information obtained from a selected other application (i.e., “fitbit”), where the health hub UI 3802 is displayed by the mobile health application. In this way, the mobile health application presents information from other applications without navigating away from the mobile health application. For example, the mobile health application presents metrics (e.g., “high-level” or “summary” metrics) from a selected application without leaving the mobile health application.

Having described example procedures in accordance with one or more implementations, consider now an example system and device that can be utilized to implement the various techniques described herein.

### Example System and Device for Providing User Interaction Policies

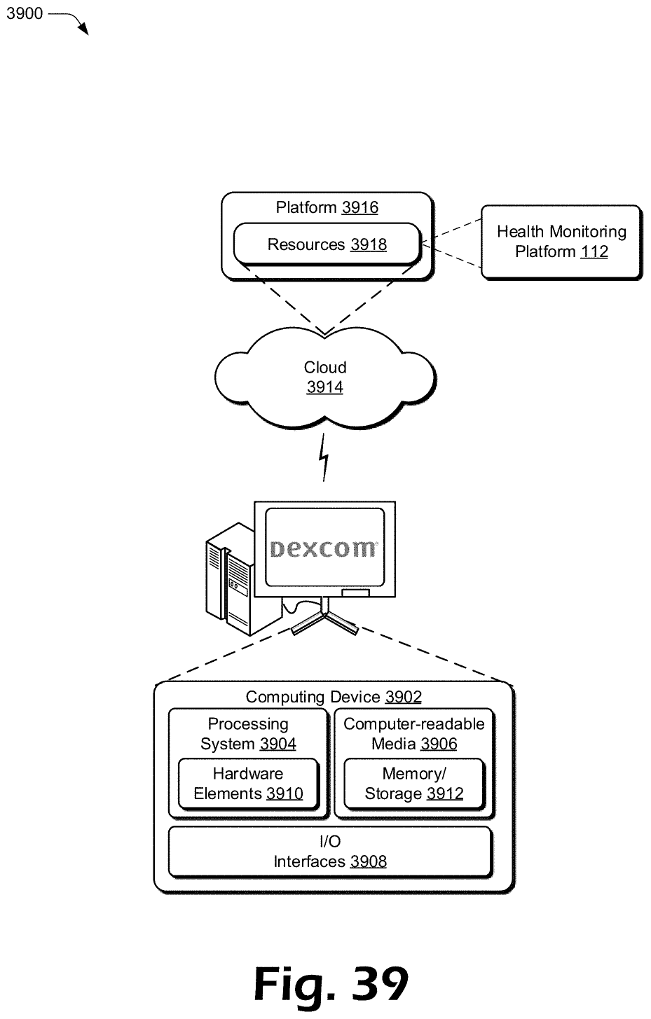

FIG. 39 illustrates an example system generally at 3900 that includes an example computing device 3902 that is representative of one or more computing systems and/or devices that may implement the various techniques described herein. This is illustrated through inclusion of the health monitoring platform 112. The computing device 3902 may be, for example, a server of a service provider, a device associated with a client (e.g., a client device), an on-chip system, and/or any other suitable computing device or computing system.

The example computing device 3902 as illustrated includes a processing system 3904, one or more computer-readable media 3906, and one or more I/O interfaces 3908 that are communicatively coupled, one to another. Although not shown, the computing device 3902 may further include a system bus or other data and command transfer system that couples the various components, one to another. A system bus can include any one or combination of different bus structures, such as a memory bus or memory controller, a peripheral bus, a universal serial bus, and/or a processor or local bus that utilizes any of a variety of bus architectures. A variety of other examples are also contemplated, such as control and data lines.

The processing system 3904 is representative of functionality to perform one or more operations using hardware. Accordingly, the processing system 3904 is illustrated as including hardware elements 3910 that may be configured as processors, functional blocks, and so forth. This may include implementation in hardware as an application specific integrated circuit or other logic device formed using one or more semiconductors. The hardware elements 3910 are not limited by the materials from which they are formed or the processing mechanisms employed therein. For example, processors may be comprised of semiconductor(s) and/or transistors (e.g., electronic integrated circuits (ICs)). In such a context, processor-executable instructions may be electronically-executable instructions.

The computer-readable media 3906 is illustrated as including memory/storage 3912. The memory/storage 3912 represents memory/storage capacity associated with one or more computer-readable media. The memory/storage component 3912 may include volatile media (such as random access memory (RAM)) and/or nonvolatile media (such as read only memory (ROM), Flash memory, optical disks, magnetic disks, and so forth). The memory/storage component 3912 may include fixed media (e.g., RAM, ROM, a fixed hard drive, and so on) as well as removable media (e.g., Flash memory, a removable hard drive, an optical disc, and so forth). The computer-readable media 3906 may be configured in a variety of other ways as further described below.

Input/output interface(s) 3908 are representative of functionality to allow a user to enter commands and information to computing device 3902, and also allow information to be presented to the user and/or other components or devices using various input/output devices. Examples of input devices include a keyboard, a cursor control device (e.g., a mouse), a microphone, a scanner, touch functionality (e.g., capacitive or other sensors that are configured to detect physical touch), a camera (e.g., which may employ visible or non-visible wavelengths such as infrared frequencies to recognize movement as gestures that do not involve touch), and so forth. Examples of output devices include a display device (e.g., a monitor or projector), speakers, a printer, a network card, tactile-response device, and so forth. Thus, the computing device 3902 may be configured in a variety of ways as further described below to support user interaction.

Various techniques may be described herein in the general context of software, hardware elements, or program modules. Generally, such modules include routines, programs, objects, elements, components, data structures, and so forth that perform particular tasks or implement particular abstract data types. The terms “module,” “functionality,” and “component” as used herein generally represent software, firmware, hardware, or a combination thereof. The features of the techniques described herein are platform-independent, meaning that the techniques may be implemented on a variety of commercial computing platforms having a variety of processors.

An implementation of the described modules and techniques may be stored on or transmitted across some form of computer-readable media. The computer-readable media may include a variety of media that may be accessed by the computing device 3902. By way of example, and not limitation, computer-readable media may include “computer-readable storage media” and “computer-readable signal media.”

“Computer-readable storage media” may refer to media and/or devices that enable persistent and/or non-transitory storage of information in contrast to mere signal transmission, carrier waves, or signals per se. Thus, computer-readable storage media refers to non-signal bearing media. The computer-readable storage media includes hardware such as volatile and non-volatile, removable and non-removable media and/or storage devices implemented in a method or technology suitable for storage of information such as computer readable instructions, data structures, program modules, logic elements/circuits, or other data. Examples of computer-readable storage media may include, but are not limited to, RAM, ROM, EEPROM, flash memory or other memory technology, CD-ROM, digital versatile disks (DVD) or other optical storage, hard disks, magnetic cassettes, magnetic tape, magnetic disk storage or other magnetic storage devices, or other storage device, tangible media, or article of manufacture suitable to store the desired information and which may be accessed by a computer.

“Computer-readable signal media” may refer to a signal-bearing medium that is configured to transmit instructions to the hardware of the computing device 3902, such as via a network. Signal media typically may embody computer readable instructions, data structures, program modules, or other data in a modulated data signal, such as carrier waves, data signals, or other transport mechanism. Signal media also include any information delivery media. The term “modulated data signal” means a signal that has one or more of its characteristics set or changed in such a manner as to encode information in the signal. By way of example, and not limitation, communication media include wired media such as a wired network or direct-wired connection, and wireless media such as acoustic, RF, infrared, and other wireless media.

As previously described, hardware elements 3910 and computer-readable media 3906 are representative of modules, programmable device logic and/or fixed device logic implemented in a hardware form that may be employed in some embodiments to implement at least some aspects of the techniques described herein, such as to perform one or more instructions. Hardware may include components of an integrated circuit or on-chip system, an application-specific integrated circuit (ASIC), a field-programmable gate array (FPGA), a complex programmable logic device (CPLD), and other implementations in silicon or other hardware. In this context, hardware may operate as a processing device that performs program tasks defined by instructions and/or logic embodied by the hardware as well as a hardware utilized to store instructions for execution, e.g., the computer-readable storage media described previously.

Combinations of the foregoing may also be employed to implement various techniques described herein. Accordingly, software, hardware, or executable modules may be implemented as one or more instructions and/or logic embodied on some form of computer-readable storage media and/or by one or more hardware elements 3910. The computing device 3902 may be configured to implement particular instructions and/or functions corresponding to the software and/or hardware modules. Accordingly, implementation of a module that is executable by the computing device 3902 as software may be achieved at least partially in hardware, e.g., through use of computer-readable storage media and/or hardware elements 3910 of the processing system 3904. The instructions and/or functions may be executable/operable by one or more articles of manufacture (for example, one or more computing devices 3902 and/or processing systems 3904) to implement techniques, modules, and examples described herein.

The techniques described herein may be supported by various configurations of the computing device 3902 and are not limited to the specific examples of the techniques described herein. This functionality may also be implemented all or in part through use of a distributed system, such as over a “cloud” 3914 via a platform 3916 as described below.

The cloud 3914 includes and/or is representative of a platform 3916 for resources 3918. The platform 3916 abstracts underlying functionality of hardware (e.g., servers) and software resources of the cloud 3914. The resources 3918 may include applications and/or data that can be utilized while computer processing is executed on servers that are remote from the computing device 3902. Resources 3918 can also include services provided over the Internet and/or through a subscriber network, such as a cellular or Wi-Fi network.

The platform 3916 may abstract resources and functions to connect the computing device 3902 with other computing devices. The platform 3916 may also serve to abstract scaling of resources to provide a corresponding level of scale to encountered demand for the resources 3918 that are implemented via the platform 3916. Accordingly, in an interconnected device embodiment, implementation of functionality described herein may be distributed throughout the system 3900. For example, the functionality may be implemented in part on the computing device 3902 as well as via the platform 3916 that abstracts the functionality of the cloud 3914.

### EXAMPLE EMBODIMENTS

Embodiment 1: A non-transitory computer readable medium comprising instructions that, when executed by at least one processor, cause the at least one processor to perform a method including: dividing a plurality of users into an exploration subset of users and an exploitation subset of users; randomly assigning at least one user interaction policy to each of the exploration subset of users; and determining at least one user interaction policy for each of the exploitation subset of users using one or more contextual models trained using contextual data corresponding to the exploitation subset of users, wherein the contextual data corresponding to the exploitation subset of users comprises at least some of a first set of contextual profiles and a second set of contextual profiles.

Embodiment 2: the non-transitory computer readable medium of Embodiment 1, wherein the exploration-exploitation phase is further performed by: receiving user feedback telemetry from the exploitation subset of users, wherein the feedback telemetry provides information regarding effectiveness of the at least one user interaction policy assigned to each user of the exploitation subset of users.

Embodiment 3: The non-transitory computer readable medium of Embodiment 2, wherein at least one of the one or more imputation models or at least one of the contextual models is retrained using the user feedback telemetry.

Embodiment 4: The non-transitory computer readable medium of Embodiment 1, wherein the exploration-exploitation phase is further performed by: measuring outcomes associated with the exploitation subset of users, wherein the measured outcomes provide information regarding effectiveness of the at least one user interaction policy assigned to each user of the exploitation subset of users.

Embodiment 5: The non-transitory computer readable medium of Embodiment 4, wherein at least one of the contextual models is retrained using the measured outcomes.

Embodiment 6: The non-transitory computer readable medium of Embodiment 1, wherein the method further comprises: collecting contextual data for a first subset of the plurality of users; generating the first set of contextual profiles for the first subset of the plurality of users based on the collected contextual data; determining that contextual data for a second subset of the plurality of users is incomplete or not available; training one or more imputation models based on the contextual data for the first subset of the plurality of users to develop the contextual data for the second subset of the plurality of users; generating the contextual data for the second subset of the plurality of users using the one or more imputation models; and generating the second set of contextual profiles for the second subset of the plurality of users based on the generated contextual data for the second subset of the plurality of users.

Embodiment 7: The non-transitory computer readable medium of Embodiment 6, wherein: the contextual data for the first subset of the plurality of users corresponds to psychographic data for the first subset of the plurality of users; the first set of contextual profiles for the first subset of the plurality of users corresponds to a first set of psychographic profiles for the first subset of the plurality of users; the contextual data for the second subset of the plurality of users corresponds to psychographic data for the second subset of the plurality of users; and the second set of contextual profiles for the second subset of the plurality of users corresponds to a second set of psychographic profiles for the second subset of the plurality of users.

Embodiment 8: The non-transitory computer readable medium of Embodiment 6, wherein determining that contextual data for the second subset of the plurality of users is incomplete or not available comprises: requesting the contextual data from the second subset of the plurality of users; and determining that at least one of (1) some of the second subset of the plurality of users provided incomplete data in response to the requesting or (2) some of the second subset of the plurality of users provided no data in response to the requesting.

Embodiment 9: A non-transitory computer readable medium comprising instructions that, when executed by at least one processor, cause the at least one processor to perform a method including: collecting contextual data for a first subset of a plurality of users; generating a first set of contextual profiles for the first subset of the plurality of users based on the collected contextual data; determining that contextual data for a second subset of the plurality of users is incomplete or not available; training one or more imputation models based on the contextual data for the first subset of the plurality of users to develop the contextual data for the second subset of the plurality of users; generating the contextual data for the second subset of the plurality of users using the one or more imputation models; and generating the second set of contextual profiles for the second subset of the plurality of users based on the generated contextual data for the second subset of the plurality of users.

Embodiment 10: The non-transitory computer readable medium of Embodiment 9, wherein: the contextual data for the first subset of the plurality of users corresponds to psychographic data for the first subset of the plurality of users; the first set of contextual profiles for the first subset of the plurality of users corresponds to a first set of psychographic profiles for the first subset of the plurality of users; the contextual data for the second subset of the plurality of users corresponds to psychographic data for the second subset of the plurality of users; and the second set of contextual profiles for the second subset of the plurality of users corresponds to a second set of psychographic profiles for the second subset of the plurality of users.

Embodiment 11: The non-transitory computer readable medium of Embodiment 9, wherein determining that contextual data for the second subset of the plurality of users is incomplete or not available comprises: requesting the contextual data from the second subset of the plurality of users; and determining that at least one of (1) some of the second subset of the plurality of users provided incomplete data in response to the requesting or (2) some of the second subset of the plurality of users provided no data in response to the requesting.

Embodiment 12: The non-transitory computer readable medium of Embodiment 9, wherein the method further comprises: dividing the plurality of users into an exploration subset of users and an exploitation subset of users; randomly assigning at least one user interaction policy to each of the exploration subset of users; and determining at least one user interaction policy for each of the exploitation subset of users using one or more contextual models trained using contextual data corresponding to the exploitation subset of users, wherein the contextual data corresponding to the exploitation subset of users comprises at least some of the first set of contextual profiles and the second set of contextual profiles.

Embodiment 13: the non-transitory computer readable medium of Embodiment 12, wherein the exploration-exploitation phase is further performed by: receiving user feedback telemetry from the exploitation subset of users, wherein the feedback telemetry provides information regarding effectiveness of the at least one user interaction policy assigned to each user of the exploitation subset of users.

Embodiment 14: The non-transitory computer readable medium of Embodiment 13, wherein at least one of the one or more imputation models or at least one of the contextual models is retrained using the user feedback telemetry.

Embodiment 15: The non-transitory computer readable medium of Embodiment 12, wherein the exploration-exploitation phase is further performed by: measuring outcomes associated with the exploitation subset of users, wherein the measured outcomes provide information regarding effectiveness of the at least one user interaction policy assigned to each user of the exploitation subset of users.

Embodiment 16: The non-transitory computer readable medium of Embodiment 15, wherein at least one of the contextual models is retrained using the measured outcomes.

What is claimed is:

1. A non-transitory computer readable medium comprising instructions that, when executed by at least one processor, cause the at least one processor to perform a method including:
collecting contextual data for a first subset of a plurality of users;
generating a first set of contextual profiles for the first subset of the plurality of users based on the collected contextual data;
determining that contextual data for a second subset of the plurality of users is incomplete or not available;
training one or more imputation models based on the contextual data for the first subset of the plurality of users to develop the contextual data for the second subset of the plurality of users;
generating the contextual data for the second subset of the plurality of users using the one or more imputation models; and
generating a second set of contextual profiles for the second subset of the plurality of users based on the generated contextual data for the second subset of the plurality of users.

2. The non-transitory computer readable medium of claim 1, wherein:
the contextual data for the first subset of the plurality of users corresponds to psychographic data for the first subset of the plurality of users;
the first set of contextual profiles for the first subset of the plurality of users corresponds to a first set of psychographic profiles for the first subset of the plurality of users;
the contextual data for the second subset of the plurality of users corresponds to psychographic data for the second subset of the plurality of users; and
the second set of contextual profiles for the second subset of the plurality of users corresponds to a second set of psychographic profiles for the second subset of the plurality of users.

3. The non-transitory computer readable medium of claim 1, wherein the method further comprises performing an exploration-exploitation phase by:
dividing the plurality of users into an exploration subset of users and an exploitation subset of users;
randomly assigning at least one user interaction policy to each of the exploration subset of users; and
determining at least one user interaction policy for each of the exploitation subset of users using one or more contextual models trained using contextual data corresponding to the exploitation subset of users, wherein the contextual data comprises at least some of the first set of contextual profiles and the second set of contextual profiles.

4. The non-transitory computer readable medium of claim 3, wherein the exploration-exploitation phase is further performed by:
receiving user feedback telemetry from the exploitation subset of users, wherein the feedback telemetry provides information regarding effectiveness of the at least one user interaction policy assigned to each user of the exploitation subset of users.

5. The non-transitory computer readable medium of claim 4, wherein at least one of the one or more imputation models or at least one of the contextual models is retrained using the user feedback telemetry.

6. The non-transitory computer readable medium of claim 3, wherein the exploration-exploitation phase is further performed by:
measuring outcomes associated with the exploitation subset of users, wherein the measured outcomes provide information regarding effectiveness of the at least one user interaction policy assigned to each user of the exploitation subset of users.

7. The non-transitory computer readable medium of claim 6, wherein at least one of the contextual models is retrained using the measured outcomes.

8. The non-transitory computer readable medium of claim 1, wherein at least one of the contextual models is a contextual multi-armed bandit model.

9. A method, comprising:
collecting contextual data for a first subset of a plurality of users;
generating a first set of contextual profiles for the first subset of the plurality of users based on the collected contextual data;
determining that contextual data for a second subset of the plurality of users is incomplete or not available;
training one or more imputation models based on the contextual data for the first subset of the plurality of users to develop the contextual data for the second subset of the plurality of users;
generating the contextual data for the second subset of the plurality of users using the one or more imputation models; and
generating a second set of contextual profiles for the second subset of the plurality of users based on the generated contextual data for the second subset of the plurality of users.

10. The method of claim 9, wherein:
the contextual data for the first subset of the plurality of users corresponds to psychographic data for the first subset of the plurality of users;
the first set of contextual profiles for the first subset of the plurality of users corresponds to a first set of psychographic profiles for the first subset of the plurality of users;
the contextual data for the second subset of the plurality of users corresponds to psychographic data for the second subset of the plurality of users; and
the second set of contextual profiles for the second subset of the plurality of users corresponds to a second set of psychographic profiles for the second subset of the plurality of users.

11. The method of claim 9, wherein the method further comprises performing an exploration-exploitation phase by:
dividing the plurality of users into an exploration subset of users and an exploitation subset of users;
randomly assigning at least one user interaction policy to each of the exploration subset of users; and
determining at least one user interaction policy for each of the exploitation subset of users using one or more contextual models trained using contextual data corresponding to the exploitation subset of users, wherein the contextual data comprises at least some of the first set of contextual profiles and the second set of contextual profiles.

12. The method of claim 11, wherein the exploration-exploitation phase is further performed by:
receiving user feedback telemetry from the exploitation subset of users, wherein the feedback telemetry provides information regarding effectiveness of the at least one user interaction policy assigned to each user of the exploitation subset of users.

13. The method of claim 12, wherein at least one of the one or more imputation models or at least one of the contextual models is retrained using the user feedback telemetry.

14. The method of claim 11, wherein the exploration-exploitation phase is further performed by:
measuring outcomes associated with the exploitation subset of users, wherein the measured outcomes provide information regarding effectiveness of the at least one user interaction policy assigned to each user of the exploitation subset of users.

15. The method of claim 14, wherein at least one of the contextual models is retrained using the measured outcomes.

16. The method of claim 9, wherein at least one of the contextual models is a contextual multi-armed bandit model.

17. A computing system, comprising:
one or more memories comprising executable instructions;
one or more processors in data communication with the one or more memories and configured to execute the instructions to:
collect contextual data for a first subset of a plurality of users;
generate a first set of contextual profiles for the first subset of the plurality of users based on the collected contextual data;
determine that contextual data for a second subset of the plurality of users is incomplete or not available;
train one or more imputation models based on the contextual data for the first subset of the plurality of users to develop the contextual data for the second subset of the plurality of users;
generate the contextual data for the second subset of the plurality of users using the one or more imputation models; and
generate a second set of contextual profiles for the second subset of the plurality of users based on the generated contextual data for the second subset of the plurality of users.

18. The computing system of claim 17, wherein:
the contextual data for the first subset of the plurality of users corresponds to psychographic data for the first subset of the plurality of users;
the first set of contextual profiles for the first subset of the plurality of users corresponds to a first set of psychographic profiles for the first subset of the plurality of users;
the contextual data for the second subset of the plurality of users corresponds to psychographic data for the second subset of the plurality of users; and
the second set of contextual profiles for the second subset of the plurality of users corresponds to a second set of psychographic profiles for the second subset of the plurality of users.

19. The computing system of claim 17, wherein the processor is further configured to perform an exploration-exploitation phase, and wherein the processor being configured to perform the exploration-exploitation phase comprises the processor being configured to:
divide the plurality of users into an exploration subset of users and an exploitation subset of users;
randomly assign at least one user interaction policy to each of the exploration subset of users; and
determine at least one user interaction policy for each of the exploitation subset of users using one or more contextual models trained using contextual data corresponding to the exploitation subset of users, wherein the contextual data comprises at least some of the first set of contextual profiles and the second set of contextual profiles.

20. The computing system of claim 3, wherein the processor is further configured to perform an exploration-exploitation phase, and wherein the processor being configured to perform the exploration-exploitation phase comprises the processor being further configured to:
receive user feedback telemetry from the exploitation subset of users, wherein the feedback telemetry provides information regarding effectiveness of the at least one user interaction policy assigned to each user of the exploitation subset of users.
